(19) United States
(12) Patent Application Publication
(10) Pub. No.: US 2022/0234981 A1
(43) Pub. Date: Jul. 28, 2022

(54) EXTRACTION AND PURIFICATION OF NATURAL FERULATE AND COUMARATE FROM BIOMASS

(71) Applicant: Spero Renewables, LLC, Goleta, CA (US)

(72) Inventors: Ian M. KLEIN, Goleta, CA (US); John STAIR, Goleta, CA (US)

(21) Appl. No.: 17/627,468

(22) PCT Filed: Jul. 16, 2020
(86) PCT No.: PCT/US2020/042392

§ 371 Date: Jan. 14, 2022

Related U.S. Application Data
(60) Provisional application No. 62/874,592, filed on Jul. 16, 2019.

(51) Int. Cl. C07C 51/09; C07C 51/43; C07C 51/47; C07C 67/56; C11B 1/02; C11C 1/04

(52) U.S. Cl. CPC C07C 51/09; C07C 51/43; C11C 1/04; C07C 67/56; C11B 1/02; C07C 51/47

(57) ABSTRACT

A process for a reactive extraction and subsequent purification of organic molecules from biomass comprises extracting one or more products from the biomass using an extraction solvent to solvate the products, contacting the biomass with a reactant during the extracting, recovering the one or more products, performing ultrafiltration to remove impurities from the one or more products to produce a filtered extract, extracting oils in the filtered extract using adsorption to produce a de-oiled extract, performing transesterification or hydrolysis on the de-oiled extract, and performing adsorptive purification on the ferulic acid, coumaric acid, ferulate, coumarate, or a combination thereof. The one or more products comprise extracted organic molecules comprising a ferulate or a coumarate, and the one or more products are separated from the biomass as a liquid extract.

### CROSS-REFERENCE TO RELATED APPLICATIONS

This application claims the benefit of U.S. Provisional Application No. 62/874,592 filed on Jul. 16, 2019 and entitled “Extraction and Purification of Natural Ferulate and Coumarate from Biomass,” which is incorporated herein by reference in its entirety for all purposes.

### STATEMENT REGARDING GOVERNMENTALLY SPONSORED RESEARCH OR DEVELOPMENT

Not applicable.

### BACKGROUND

Consumer demands for natural products and ingredients have driven the industry to seek new sources of natural vanilla, while vanilla derived from synthetic, non-natural sources such as petrochemicals and eugenol have seen a decrease in demand. Carbon 13 NMR of vanillin (the active molecule in vanilla flavoring) is used to determine the source of vanillin by the ratio of ¹³C and ¹²C at the eight carbons of the vanillin molecule, with the carbons of the aldehyde and the methoxy group determined to be the most important. Synthetic sources such as guaiacol can be discriminated from natural vanillin sourced from vanilla beans and ferulic acid, with thresholds dependent on the experimental setup.

Much of the supply of natural ferulic acid is extracted during the processing of rice bran oil. Ferulic acid is also contained in agricultural biomass such as miscanthus, corn byproducts (fiber, bran, stover, fines, gluten feed, etc.), rice, wheat, beets, beet fiber, beet pulp, and other crops. Typical methods for extracting ferulic acid include alkali extraction and enzymatic processes. Alternatively, ferulic acid is extracted as several ferulic acid phytosterol esters such or γ-oryzanol. Excess alcohol extraction has been used to remove lignin from biomass, and similar extractions have been used to extract ferulic acid esters. For example, in the presence of methanol, the extract contains methyl ferulate (methyl 3-(4-hydroxy-3-methoxyphenyl)acrylate), while in the presence of ethanol the extract contains ethyl ferulate (ethyl 3-(4-hydroxy-3-methoxyphenyl)acrylate). Additional processes for recovering ferulic acid from biomass are described in WO2018/195422 to Abu-Omar, et al., which is incorporated herein in its entirety by reference.

Extractions of ferulate esters using distinct and separate reaction and extraction steps have been demonstrated previously such as in U.S. Pat. No. 5,843,499 (1998), showing significantly lower ferulate ester yields than the ferulate ester yields presented herein.

### SUMMARY

In some aspects, a process for a reactive extraction and subsequent purification of organic molecules from biomass comprises extracting one or more products from the biomass using an extraction solvent to solvate the products, contacting the biomass with a reactant during the extracting, recovering the one or more products, performing ultrafiltration or nanofiltration to remove impurities from the one or more products to produce a filtered extract, extracting oils in the filtered extract using adsorption to produce a de-oiled extract, performing transesterification or hydrolysis on the de-oiled extract, and performing adsorptive purification on the ferulic acid, coumaric acid, ferulate, coumarate, or a combination thereof. The one or more products comprise extracted organic molecules comprising a ferulate or a coumarate, and the one or more products are separated from the biomass as a liquid extract. The ferulate or the coumarate can be reacted in a transesterification or hydrolysis to produce ferulic acid, coumaric acid, ferulate, coumarate, or a combination thereof. Ferulic acid, coumaric acid, ferulate, coumarate, or a combination thereof are purified to produce one or more purified products.

In some aspects, a process to purify biomass extracts containing ferulate or coumarate comprises: obtaining a biomass extract from biomass; and performing filtration on the biomass extract to remove large molecular weight impurities from the biomass extract. The filtration uses ultrafiltration or nanofiltration.

In some aspects, a process to selectively remove oil from biomass extracts comprises obtaining a biomass extract, and performing liquid-solid adsorption to remove at least a portion of the one or more oils onto a solid phase adsorbent to produce a purified product. The biomass extract is obtained from biomass, and wherein the biomass extract contains a ferulate or a coumarate and one or more oils.

In some aspects, a chromatographic adsorption process to separate products from a biomass extract solution comprises providing a biomass extract, and performing chromatographic adsorption on the products. The biomass extract comprises the products comprising ferulates, coumarates, and oils. The chromatographic adsorption occurs in a simulated moving bed chromatography system used to separate products and other biomass extractives into substantially pure fractions.

These and other features will be more clearly understood from the following detailed description taken in conjunction with the accompanying claims.

### BRIEF DESCRIPTION OF THE DRAWINGS

For a more complete understanding of the present disclosure and the advantages thereof, reference is now made to the following brief description, taken in connection with the accompanying drawings and detailed description, wherein like reference numerals represent like parts.

FIG. 1 illustrates a flow chart showing steps for the extraction and purification of ferulate and coumarate according to some embodiments.

FIG. 2 illustrates a flow chart showing a process to produce solid ferulic acid from biomass extractives according to some embodiments.

FIG. 3 illustrates another flow chart showing a process to produce solid ferulic acid from biomass extractives according to some embodiments.

FIG. 4A represents the structure of an exemplary ferulate.

FIG. 4B represents the structure of an exemplary coumarate.

FIG. 5 illustrates the total ferulate yield based on the reaction as described with respect to example 6.

FIG. 6 illustrates the total ferulate yield based on the reaction as described with respect to example 5.

FIG. 7 illustrates a combined yield of ferulate and coumarate based on the reaction as described with respect to example 7.

FIG. 8 shows a HPLC chromatogram for chemical standards of ferulic acid, coumaric acid, methyl ferulate, methyl coumarate, and ethyl ferulate.

FIG. 9 shows a simulated moving bed chromatography system used to purify ferulates according to some embodiments.

FIG. 10 depicts a flow chart of an integrated process to produce ferulic acid from biomass using simulated moving bed chromatography as a purification according to some embodiments.

FIG. 11 depicts a flow chart of another integrated process to produce ferulic acid from biomass using ion exchange as a purification according to some embodiments.

FIG. 12 illustrates a chromatogram as analyzed by HPLC of the yield of ethyl ferulate in a filtrate as described with respect to example 2.

FIG. 13 illustrates a chromatogram as analyzed by HPLC of the yield of methyl ferulate as described with respect to example 3.

FIG. 14 illustrates a chart showing the hourly extraction of methyl ferulate and methyl coumarate as described with respect to example 3.

FIG. 15 illustrates the HPLC chromatogram of the final reaction products according to the reaction as described with respect to example 6.

FIG. 16 illustrates UV-VIS chromatogram as described with respect to example 13.

FIG. 17 illustrates UV absorption data and chromatography as described with respect to example 14.

FIG. 18 illustrates UV absorption data and collection data as described with respect to example 15.

FIG. 19 is a table showing the fractions of various compounds provided in the process as described in example 15.

FIG. 20 the HPLC chromatogram of the final reaction products according to the reaction as described with respect to example 16.

### DETAILED DESCRIPTION

It is understood at the outset that although an illustrative implementation of one or more embodiments are provided below, the disclosed systems and methods may be implemented using any number of techniques, whether currently known or in existence. The disclosure should in no way be limited to the illustrative implementations, drawings, and techniques illustrated herein below, including the exemplary designs and implementations illustrated and described herein, but may be modified within the scope of the appended claims along with their full scope of equivalents. Thus, while multiple embodiments are disclosed, still other embodiments will become apparent to those skilled in the art from the following detailed description. As will be apparent, certain embodiments, as disclosed herein, are capable of modifications in various aspects without departing from the spirit and scope of the claims as presented herein. Accordingly, the detailed description herein below is to be regarded as illustrative in nature and not restrictive.

Ferulic acid (3-(4-hydroxy-3-methoxy-phenyl)prop-2-enoic acid, 3-(4-hydroxy-3-methoxyphenyl)acrylic acid) is a powerful anti-oxidant used in consumer products and pharmaceuticals. Ferulic acid is also used to produce natural vanilla flavoring. The flavor and fragrance industry transforms ferulic acid to natural vanilla flavoring (vanillin) using enzymatic processes. Natural vanillin produced from natural ferulic acid is of particular importance due to the volatility, high cost, and scarcity of natural vanilla extract derived from vanilla beans.

The extracted ferulate and coumarate can be regarded as natural, for example following European 1334/2008 and US Food and Drug administration (FDA) 21CFR101.22 regulations regarding natural labeling. The extracted ferulate can be hydrolyzed to ferulic acid and isolated as a pure, crystalline solid or as an aqueous solution. Additional cinnamic acids, sugars, oils, and fatty acids are also extracted during the process and can be isolated as coproducts.

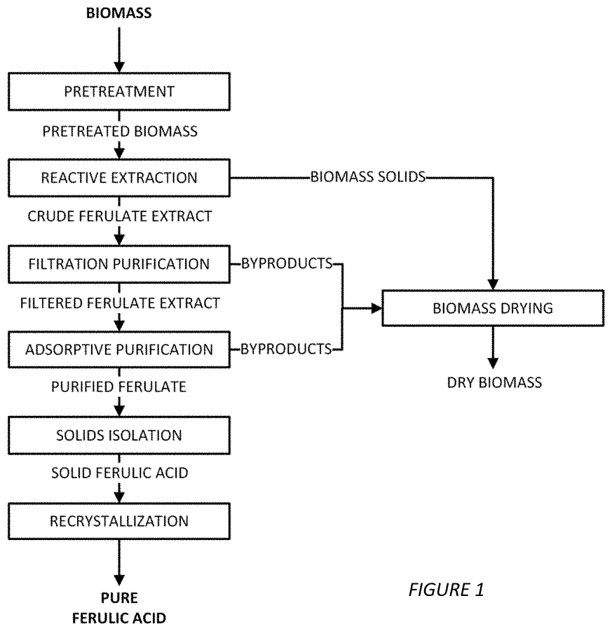

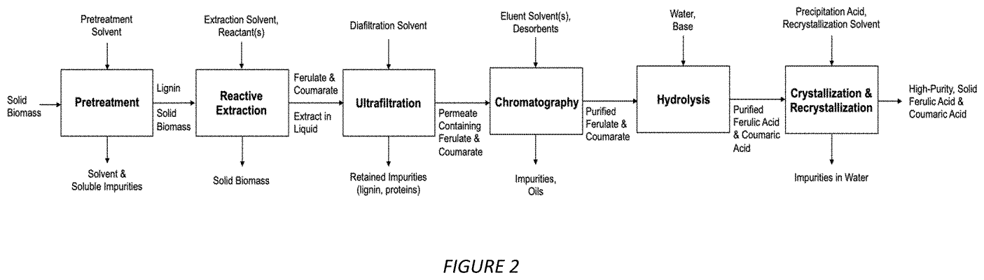

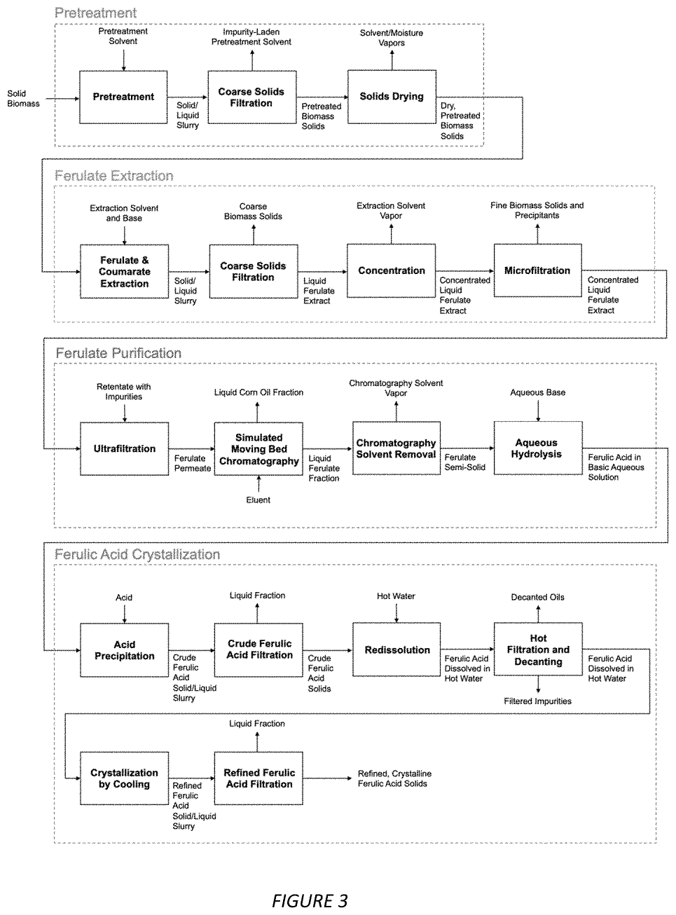

This application describes a process for producing ferulic acid from biomass, where said process may comprise any number of the steps summarized herein, where such steps can comprise pretreatment of agricultural biomass, reactive extraction of ferulate (ferulic acid esters of the variety methyl-, ethyl-, propyl-, butyl-, or any variation thereof) and coumarate, purification of the resulting ferulate and coumarate using filtration methods and chromatography, conversion of ferulate and coumarate to ferulic acid and coumaric acid, purification of ferulic acid by ion exchange, and crystallization and recovery of solid ferulic acid and coumaric acid. FIG. 1 shows a generalized depiction of steps that can be included as part of the present disclosure. There are several specific configurations of key processing steps that can be used to produce ferulic acid from biomass. One example configuration is depicted in FIG. 2. FIG. 2 shows an overview of these key processing steps in the ferulic acid production process. In general, the process can be subdivided into four major steps. In the first step, agricultural biomass, such as miscanthus and/or corn byproducts, can be pretreated to remove certain extractives such as sugars and proteins. In the second step the reactive extraction of ferulate and coumarate can be performed. The third step of the process can include purification of the resulting ferulate and coumarate using a combination of microfiltration, ultrafiltration, nanofiltration, and/or chromatography. In the fourth and final step, ferulate and coumarate can be hydrolyzed to ferulic acid and coumaric acid, purified by ion-exchange, crystallized, and recovered as a high-purity solid. Another example process is shown in FIG. 3, where simulated moving bed chromatography is used to purify ferulate. FIG. 3 shows the major steps including pretreatment, extraction, purification, and crystallization, and their constituent subprocesses as described further herein.

The first step can include a process to pretreat biomass. The pretreatment process can begin with a pretreatment step to remove certain impurities from agricultural biomass. Any suitable biomass can be used as a starting feedstock. Various exemplary biomass feedstocks include, but are not limited to, miscanthus, corn bran, corn fiber, corn gluten feed, distillers' grain, corn stover, corn gluten meal, beet fiber, rice hulls, and/or other agricultural residues containing ferulate linkages. Wheat bran or other wheat derived materials can also be used as a feedstock for the process described herein. During the pretreatment step, the biomass is heated in the presence of a solvent to remove extractives including but not limited to sugars and proteins. The pretreatment solvent consists of 0-100% water and pure components or mixtures of aliphatic alcohols (methanol, ethanol, n-propanol, iso-propyl alcohol, n-butanol, 2-butanol, tert-butanol, n-pentanol) comprising the remainder of the solvent. Acids or bases can be added to the solvent to catalyze the pretreatment removal of extractives including proteins and sugars at lower reaction temperatures than would be required without the acids or bases. Bases include any first or second group hydroxides such as sodium hydroxide or potassium hydroxide, carbonates, bicarbonates, and ammonium in concentrations of 0 to 6 N (molar equivalents of hydroxide per liter of solvent). Acids include inorganic acids, such as hydrochloric acid, phosphoric acid, and sulfuric acid, and organic acids, such as citric acid.

Various process designs can be used to carry out the pretreatment. Any suitable reactor configuration capable of contacting the biomass feedstock with the pretreatment solvent can be used. For example, the pretreatment of agricultural biomass can occur in a stirred batch reactor, continuous stirred-tank reactor (CSTR), or packed bed reactor. The pretreatment step may also be carried out in the same reactor or vessel as the subsequent reactive extraction step.

In some embodiments, the pretreatment of agricultural biomass occurs in a stirred batch reaction configuration, the biomass and pretreatment solvent can be mixed together in a pressurized vessel and sealed shut. Acid or base pretreatment aids can be added into the solvent at the beginning of the pretreatment, or added at a later time over the course of the treatment. The mass to mass ratio for solvent:biomass can be in the range of 2-30. In some embodiments, a mass to mass ratio for solvent:biomass of 10-20 can be used. In other embodiments, a mass to mass ratio for solvent:biomass of 6-20, or alternatively 6-15, can be used. The reaction atmosphere can be purged of oxygen such that the atmosphere is inert with argon, helium, nitrogen, or a mixture thereof. The reactor can be heated at a ramp rate of 50-300° C. hr⁻¹ to a dwell temperature of between 25-250° C. In some embodiments, the pretreatment reactor temperature is between about 60-100° C. The reactor can maintain the maximum reactive extraction temperature for between about 0.25-12 hours. The stirring mechanism can be operated from about 30-600 rpm. After pretreatment, the batch reactor can be cooled to room temperature. Any pressure in the reactor can be relieved to atmospheric pressure.

In some embodiments, the pretreatment of agricultural biomass occurs in a packed bed reactor. The biomass can be packed into a pressurized vessel and sealed shut. The agricultural biomass can act as the stationary bed. Heated and pressurized solvent can be pumped over the biomass, removing extractives such as protein and polysaccharides. A back pressure regulator downstream from the packed bed can be used to maintain the reactor at a steady pressure, typically between about 1-30 bar. The mass to mass ratio for solvent:biomass can be in the range of about 2:1-30:1. In some embodiments, a mass to mass ratio for solvent:biomass in the range of about 10:1-20:1 can be used. In some embodiments, the mass to mass ratio for solvent:biomass can be 5:1-20:1. The reaction atmosphere can be purged of oxygen such that the atmosphere is inert with argon, helium, nitrogen, or a mixture thereof. The reactor can be heated to a steady-state temperature of between about 25-250° C. while flowing the pretreatment solvent. In some embodiments, the pretreatment reactor temperature can be between about 60-100° C. As the used pretreatment solvent exits the packed bed, 0-100% of the stream can be recycled to the bed entrance to reduce virgin pretreatment solvent usage. The pretreatment solvent can thus make one pass through the bed or be partially or completely recycled to the bed entrance to further pretreat the biomass. The reactor can maintain the maximum reactive extraction temperature for between 0.25-12 hours. After pretreatment, the packed bed reactor can be cooled to room temperature. The reactor can be purged to atmospheric pressure and the solids can be unloaded. In some embodiments, the biomass in the reactor can be dried using a heated stream of air or inert gas before being cooled and unloaded.

In some embodiments, the pretreatment of agricultural biomass can occur in a CSTR configuration. The biomass and solvent can be pre-mixed together and continuously fed as a slurry to a stirred vessel using a slurry pump or gravity. Any suitable equipment may be used to transport the slurry, including augers. The pretreated biomass and impurity-laden pretreatment solvent can be continuously pumped out of the reactor using a slurry pump in a stream with a volumetric flowrate equal to that of the feed stream. The mass to mass ratio for solvent:biomass can be in the range of about 2:1-30:1. In some embodiments, a mass to mass ratio for solvent:biomass of about 10:1-20:1 can be used. In some embodiments, the mass to mass ratio for solvent:biomass can be 5:1-20:1. The reactor can operate at a steady-state temperature of between about 25-250° C. Preferably, the pretreatment reactor temperature is between about 60-100° C. The reactor can operate with a residence time of about 0.25-12 hours. The stirring mechanism can be operated from 30-600 rpm.

The solid/liquid post pretreatment slurry can be removed from the reactor either by pump or by gravity. The pretreated solid biomass is separated from the pretreatment liquid using methods including but not limited to screening, sieving, centrifugation, filtration, and combinations thereof. The solid residue can be rinsed with clean pretreatment solvent using 0-150% the volume of the filtered solids. The solid residue can be rinsed in one or multiple stages, with or without solid-liquid separation steps between each rinsing stage. The wet solids can optionally be pressed to further remove solvent entrained in the solids. The pressed solids can then be dried using any drying process including rotary drying, vacuum drying, rolling bed drying, fluidized bed drying, or combinations thereof. If the biomass solid was dried in the reactor, it can be unloaded and moved to the reactive extraction step. Example 1 depicts a pretreatment occurring prior to reactive extraction of biomass.

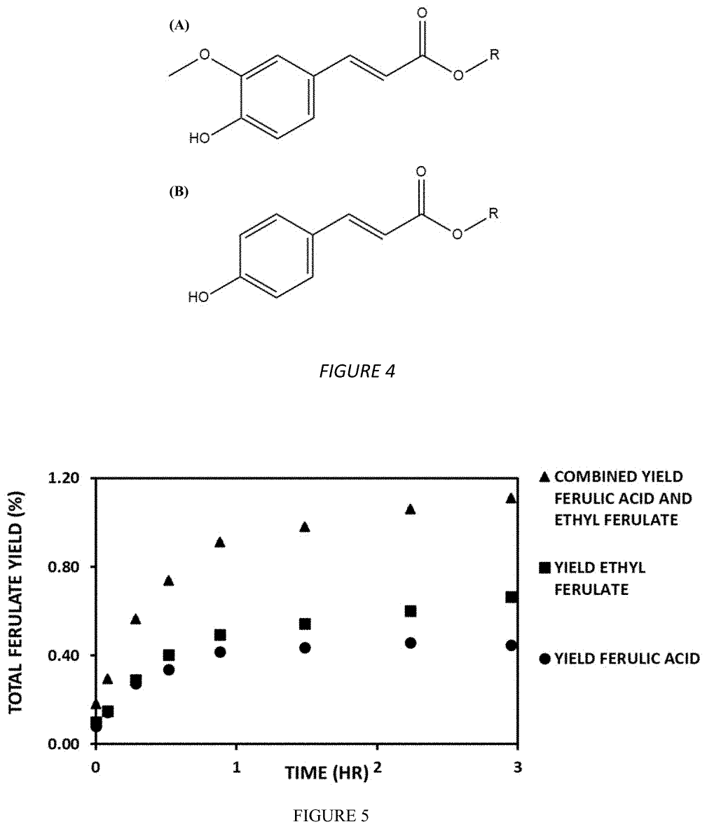

The next step can include ferulate reactive extraction. In some embodiments, a process for reactive extraction of ferulate and coumarate can include a reaction step and/or extraction step for the extraction of ferulates and coumarates from biomass, whether pretreated or unpretreated. The reaction step, which can occur prior to or simultaneously with the extraction step, can occur with a base. The extraction step can occur in the presence of a solvent. The products can comprise at least one of: ferulate (ferulic acid ester), and coumarate (coumaric acid ester). The structures of exemplary ferulates and coumarates are shown in FIG. 4. As shown in FIG. 4(A), R can represent a hydrogen or an alkyl group such that: ferulic acid: R═H, methyl ferulate: R═CH₃, ethyl ferulate: R═C₂H₅, propyl ferulate: R═C₃H₇, butyl ferulate: R═C₄H₉. As shown in FIG. 4(B), R can represent a hydrogen or an alkyl group such that: coumaric acid: R═H, methyl coumarate: R═CH₃, ethyl coumarate: R═C₂H₅, propyl coumarate: R═C₃H₇, butyl coumarate: R═C₄H₉. Coproducts are also extracted and include oils, which are typically comprised of oleate (oleic acid ester), and linoleate (linoleic acid ester).

The process can include an extraction step using an extraction solvent. In some embodiments, the extraction solvent can comprise an alcohol (e.g., an organic alcohol) along with a co-solvent such as water. While not wishing to be limited by theory, it has been noted that the identity of the ferulate is directly linked to the chosen alcohol in the solvent. For example, the use of methanol yields methyl ferulate and the use of ethanol yields ethyl ferulate. Esters of coumaric acid are simultaneously extracted. In some embodiments, the extraction solvent can comprise 0-50% water and a pure aliphatic alcohol or mixtures of aliphatic alcohols (e.g., methanol, ethanol, n-propanol, iso-propyl alcohol, n-butanol, 2-butanol, tert-butanol, n-pentanol, etc.), where the aliphatic alcohol can comprise 50-100% of the solvent. In Example 2, extraction of ferulate and coumarate in ethanol yielded ethyl ferulate and ethyl coumarate, while in Example 3, extraction of ferulate and coumarate in methanol yielded methyl ferulate and methyl coumarate. In some embodiments, the solvent may include only water and one or more aliphatic alcohols. In still other embodiments, water may not be present in the solvent, and rather 100% alcohol of the variety methanol or ethanol can be used for the solvent. In other embodiments, water can comprise 100% of the extraction solvent.

In some embodiments, a reaction step can also be carried out using a base. This reaction step can be carried out at the same time as the extraction. For example, base can be added to the solvent to enhance solubility and rates of reaction and extraction at lower reaction temperatures. Bases include any first or second group hydroxides such as sodium hydroxide or potassium hydroxide, carbonates, bicarbonates, and ammonium in concentrations of about 0 to about 1 N (molar equivalents of base per liter of solvent). For example, the concentration of the base can be between 0.01 and about 0.1 N, or between about 0.02 and about 0.06 N, or about 0.04 N. The concentration of base can also be between 0.01 and 0.5 N. In some embodiments the concentration of base can be about 0.12 N.

Various process designs can be used to carry out the extraction and/or reaction steps. Any suitable reactor configuration capable of contacting the biomass feedstock with the solvent and/or base can be used. For example, the reactive extraction of ferulates can occur in a stirred batch reactor, continuous stirred-tank reactor (CSTR), or packed bed reactor (PBR) scheme.

To improve or optimize the yield of ferulate, several variables can be manipulated, including the concentration of extraction aid, extraction solvent to biomass ratio, the reactive extraction temperature, the reactive extraction time, the specific biomass, the extraction solvent composition, and/or the reactor equipment. Several sets of operating conditions are hereinafter enumerated, but other regimes of operating conditions are possible and included as part of this disclosure.

Small amounts of water are unavoidably present as a moisture component in alcohol marketed as anhydrous. Water can comprise 0-4% of the alcohol by weight even when labeled as anhydrous. In embodiments where the extraction solvent is composed of anhydrous alcohol, small amounts of water are inevitably present in the alcohol. Biomass introduced into the extraction solvent also contains some moisture, the amount of which varies based on the extent to which the biomass is dried, and which contributes the total moisture content of a slurry of biomass and extraction alcohol. In embodiments where the total moisture content of the biomass and alcohol slurry is between 0% and 5%, the reactive extraction is typically carried out between 120° C.-170° C. with a solvent to biomass ratio of 4 to 12, with a sodium hydroxide concentration of 0.04M-0.16M, and a reaction time of 2-12 hours. In embodiments where the total moisture content of biomass and alcohol slurry is between 5% and 25%, the reactive extraction can be carried out between 60° C.-120° C. with a solvent to biomass ratio of 4 to 12, with a sodium hydroxide concentration of 0.04M-0.32M, and a reaction time of 0.25-12 hours.

While not wishing to be limited by theory, it is noted that when the reactive extraction of ferulate is carried out in the presence of water, whether the water is from moisture in the biomass or as a component of the virgin solvent, a competing side reaction can convert ferulate into an unknown decomposition product, resulting in a reduction of ferulate yield. While not wishing to be limited by theory, the side reaction has been observed to be faster: when the reaction mixture water content is higher; under higher reaction temperatures; and using higher concentrations of extraction aid. In some embodiments, where the extraction aid is a base, the concentration of the base affects the rate of decomposition. The yield of ferulate can be improved or optimized for operation with a certain water content by varying: the concentration of extraction aid; addition of the extraction aid to the reactive extraction over various times with optional addition of extraction aid throughout the reactive extraction; ratio of extraction solvent to biomass; reactive extraction temperature; reactive extraction time; specific biomass; the extraction solvent composition; and the reactor equipment design. Example 4 described herein shows that increasing the moisture content of corn fiber from 4 wt % to 12 wt % decreases the yield of ferulate (gram ferulate/gram dry corn fiber) from 1.75% to 1.2% when anhydrous ethanol was used as a solvent.

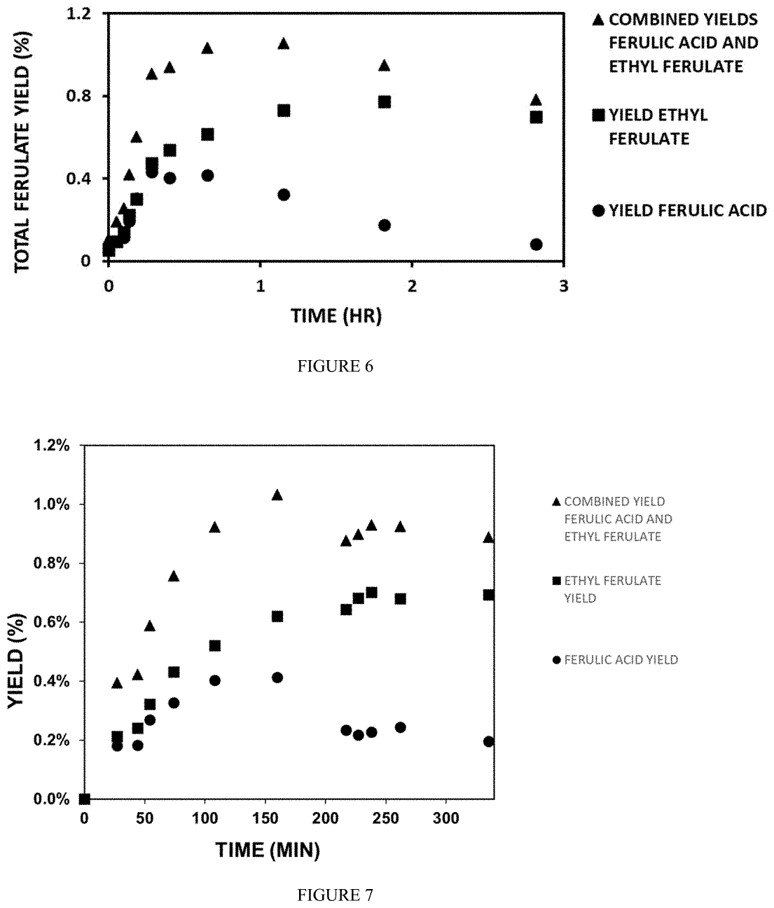

In some embodiments, the extraction solvent may be comprised of a mixture of an alcohol and water. When the extraction solvent is a mixture of an alcohol and water, the ferulate products will be a mixture of ferulate alcohol ester as well as ferulic acid. The proportion of alcohol to water in the solvent changes the ratio of ferulate ester to acid throughout the course of the reactive extraction and in the product mixture. Some or all of the total solution water content can come from moisture in the biomass as the biomass solids are incorporated into the reactive extraction mixture. In some embodiments, the extraction solvent can be a mixture of ethanol and water. In some embodiments, the extraction solvent is comprised of 80-96 wt % ethanol with the remaining 4-20 wt % consisting of water. In some embodiments, the extraction solvent consists of 87 wt % ethanol and 13 wt % water and the reactive extraction will yield a mixture of ethyl ferulate and ferulic acid in similar proportions, as further described in Example 5. In some embodiments where the extraction solvent consists of mixtures of ethanol and water and the extraction aid is sodium hydroxide, certain trends in ferulate yield are observed. One observed trend was that when the concentration of sodium hydroxide is held constant and the reaction temperature is increased, increased reaction temperatures causes degradation of ferulate reaction products over time. This trend is observed in the comparison of Example 5 and Example 6. In both Example 5 and Example 6, corn fiber was extracted using a solvent of 87 wt % ethanol and 13 wt % water with a sodium hydroxide concentration of 0.08 N in both examples. The reaction temperature of Example 5 was 120° C. while the reaction temperature of Example 6 was 100° C. In Example 6, where the reaction temperature was 100° C., the combined yields of ferulic acid and ethyl ferulate were observed to increase throughout the course of the reaction, and did not show a decrease of ferulic acid and ethyl ferulate over time, as illustrated in FIG. 5. The combined yields of ferulic acid and ethyl ferulate depicted in FIG. 5 indicate that the ferulic acid and ethyl ferulate products of Example 6 are stable and do not decay appreciably under the conditions studied. In contrast to Example 6, Example 5 maintained a reaction temperature of 120° C. and after an initial increase in the combined yields of ferulic acid and ethyl ferulate, the combined yields of ferulic acid and ethyl ferulate were observed to decrease over time as shown in FIG. 6.

In some embodiments where the extraction solvent consists of mixtures of ethanol and water and the extraction aid is sodium hydroxide, certain trends in ferulate yield are observed. One observed trend is that higher concentrations of sodium hydroxide can be tolerated without significant decomposition of ferulic acid or ethyl ferulate at lower reaction temperatures, a trend that is illustrated in Example 6 and Example 7. In both Example 6 and Example 7, corn fiber was extracted using a solvent of 87 wt % ethanol and 13 wt % water with reaction temperature of 100° C. In Example 6, where the concentration of sodium hydroxide was 0.08 N, a maximum combined yield of ethyl ferulate and ferulic acid of 1.11% was observed as shown in FIG. 5. In Example 7, where the concentration of sodium hydroxide was increased 0.16 N, a very similar combined yield of ferulic acid and ethyl ferulate of 1.03% was observed as shown in FIG. 7. In contrast, significant decomposition of ferulic acid and ethyl ferulate was observed when a solvent of 87 wt % ethanol and 13 wt % water and 0.16 N sodium hydroxide was used to extract corn fiber at a reaction temperature of 120° C. or greater.

In other embodiments the biomass is corn fiber, which may be received from a corn mill or similar facility, where the fiber contains 10-70% water by mass, or 50-60% water by mass, and where the water in the corn fiber contributes to the water in the solvent of the reactive extraction when the fiber used without drying or with partial drying to remove some portion of the water contained in the corn fiber. Any number of stages or steps of pressing, centrifugation, or other liquid removal steps can optionally be used to remove water from corn fiber after receipt from a corn mill or similar facility. The pressed solids can then optionally be dried using any drying process including rotary drying, vacuum drying, rolling bed drying, fluidized bed drying, or combinations thereof before reactive extraction of ferulate.

In some embodiments, the reactive extraction of ferulate can occur in a stirred batch reactor. The biomass and extraction solvent can be mixed together in a pressurized vessel and sealed shut. Alternatively, the pretreated biomass, solvent(s), and base can be pre-mixed as a slurry and fed into the reactor using a pump or gravity. The mass to mass ratio for solvent:biomass can be in the range of 2:1-30:1. In some embodiments, the preferred mass to mass ratio for solvent:biomass of 5:1-15:1 can be used. The reaction atmosphere can be purged of oxygen such that the atmosphere is inert with argon, helium, nitrogen, or a mixture thereof. Hydrogen (0-100%) atmospheres give similar results to a purely inert atmosphere. The reactor can be heated at a ramp rate of between about 100-400° C. hr⁻¹, or between about 275-325° C. hr⁻¹ to a dwell temperature of between 100-250° C., or between about 120-160° C. In some embodiments, the amount of water in the reaction mixture resulting from the mixture of the extraction solvent and biomass can result in an upper extraction dwell temperature of 40-120° C. The reactor can maintain the maximum extraction temperature for between about 1-24 hours, or between about 3-5 hours. The extraction temperature can also be 0.1-24 hours. The stirring mechanism can be operated from about 100-600 rpm. The stirring mechanism can also be operated between 20-1000 rpm. In some embodiments where the reaction mixture is at or above the boiling point of the solution, the reactor may be cooled by flash depressurization, wherein a portion of the solvent is boiled off, thereby cooling the reaction mixture and advantageously removing a portion of the solvent.

In some embodiments, the reactive extraction of ferulate can occur in a packed bed reactor. The biomass can be packed into a pressurized vessel and sealed shut. The biomass acts as the stationary bed. Heated and pressurized extraction solvent with or without extraction aid can be pumped over the biomass, extracting ferulate, coumarate, and oils into the mobile liquid phase. The bed can also be filled with solvent to a satisfactory level, with the liquid then being held stationary during the reactive extraction, then flushed or washed with flowing solvent at the end. A back-pressure regulator downstream from the packed bed can be used to maintain the reactor at a steady pressure, typically between about 1-30 bar. The reaction atmosphere can be purged of oxygen such that the atmosphere is inert with argon, helium, nitrogen, or a mixture thereof. The reactor can be heated to a steady-state temperature of between about 100-250° C. while flowing the extraction solvent. The typical operating conditions are: a bed temperature of between about 120-160° C., a reactor pressure of between about 1-30 bar, and a solvent flow rate of between about 0.01-1 mL per minute per gram of biomass. As the used extraction solvent exits the packed bed, 0-100% of the stream can be recycled to the bed entrance to reduce virgin extraction solvent usage. The extraction solvent can thus make one pass through the bed or be partially or completely recycled to the bed entrance to extract more ferulate from the biomass. The reactor can maintain the maximum extraction temperature for between about 0.25-24 hours. After ferulate reaction and extraction, the packed bed reactor can be cooled to room temperature. The reactor can then be cooled and the flow of solvent briefly set to about 0.05-0.15 mL per minute per gram of biomass to wash the biomass bed. The solvent flow can be stopped after the bed temperature decreases to approximately 20° C. The pressure can be relieved to atmospheric pressure after the reactor reaches room temperature. The spent biomass can then be emptied from the reactor. In some embodiments where the reaction mixture is at or above the boiling point of the solution, the reactor may be cooled by flash depressurization, wherein a portion of the solvent is boiled off, thereby cooling the reaction mixture and advantageously removing a portion of the solvent. In some embodiments, the biomass in the reactor can be dried using a heated stream of air or inert gas before being cooled and unloaded.

In some embodiments, the reactive extraction of ferulate occurs in a steady-state continuous stirred-tank reactor (CSTR) configuration. The biomass and solvent can be pre-mixed together and continuously fed as a slurry to a stirred vessel using a slurry pump or gravity. The biomass and ferulate-laden solvent can be continuously pumped out of the reactor together using a slurry pump in a stream with a volumetric flowrate equal to that of the feed. The slurry of biomass and solvent can also be continuously removed from the reactor by pressure-driven flow or any other means. The mass to mass ratio for solvent:biomass can be in the range of about 2:1-30:1. In some embodiments, the mass to mass ratio for solvent:biomass of 5:1-15:1 can be used. The reactor can operate at a steady-state temperature of between about 25-250° C., or between about 120-160° C. The reactor can operate with a residence time of between about 0.25-24 hours. The stirring mechanism can be operated from 30-600 rpm. In some embodiments where the reaction mixture is at or above the boiling point of the solution, the reactor may be cooled by flash depressurization, wherein a portion of the solvent is boiled off, thereby cooling the reaction mixture and advantageously removing a portion of the solvent. In some embodiments, following the reactive extraction of ferulates and solid-liquid separation of the remaining corn fiber from the ethyl ferulate and/or ferulic acid extract liquid, a protein-containing steep liquor, which was produced from a wet corn milling process, can optionally be added to the extracted corn fiber before it is dried to make an animal feed similar in nature to corn gluten feed.

The solid/liquid slurry exiting the reactor can be removed from the reactor either by pump or by gravity. If a packed bed reactor is used for the extraction, the biomass can be unloaded as a slurry or as dried solids. If product of any reactor configuration is a slurry of extracted biomass and a liquid containing ferulates, the bulk of the liquid must be separated from the solids in a de-slurrying step. In a de-slurrying step, the extracted solid biomass can be separated from the liquid using methods including but not limited to screening, sieving, centrifugation, filtration, and combinations thereof. Further liquid can also be removed from the solids by pressing or other methods. Washing of the solids can be used to increase recovery of products contained in liquid entrained in the solids. The coarse solids can be rinsed with clean extraction solvent (with or without base) using 0-150% the volume of the filtered solids in a separate or integrated rinsing process. The wet solids can optionally be pressed to further remove solvent entrained in the solids. Any number of stages or steps of pressing, centrifugation, or other liquid removal steps can be used. The pressed solids can then be dried using any drying process including rotary drying, vacuum drying, rolling bed drying, fluidized bed drying, or combinations thereof.

In some embodiments, the biomass solids can be washed or rinsed in multiple stages. The extracted biomass, having had the majority of the extract liquid removed by any number of solid-liquid separation steps, can be washed in one or more stages. In a washing stage, clean solvent is added to the solid by spraying, percolation, or any other means. The washing liquid is then displaced from the solid either simultaneously with, or after, the addition of washing liquid. Displacement of the washing liquid can happen by centrifugation, pressing, or other methods. The mass ratio of washing liquid to extracted biomass (dry mass basis) in each stage can be in the range of 0 to 6. In some embodiments, the biomass is washed in two stages, with a per-stage mass ratio of washing liquid to extracted biomass (dry mass basis) of 0.6-1.5. An example of a reactive extraction followed by multiple biomass washing stages is described in Example 8.

Byproduct streams are produced throughout the process, and may be composed of lignin, protein, sugars, oils, salts, or other byproducts. Byproduct streams, which can be solids or solutions of water and/or solvent, can be blended into the fiber using any suitable equipment. The extracted biomass can be useful products. The extracted biomass can be used as animal feed. Blending of byproducts into the extracted fiber may be advantageous if it increases the nutritional value of the feed. In some embodiments, byproduct solid and/or streams are blended into the extracted biomass. The blended biomass is then dried using any suitable drying process, including rotary drying, vacuum drying, rolling bed drying, fluidized bed drying, any other drying process, or combinations thereof. In some embodiments where the biomass used for reactive extraction of ferulate and/or ferulic acid is corn fiber, certain byproducts from the corn milling process may advantageously be blended with the remaining corn fiber after extraction of ferulate and ferulic acid. Corn steep liquor is a concentrated liquid derived from the water that us used in the initial stage of corn treatment in the wet corn milling process. Corn steep liquor can optionally be added to corn fiber that has been extracted using the ferulate/ferulic acid removal process described herein, where the fiber, steep liquor, and/or any process byproducts are dried together to increase the nutritional value of the extracted corn fiber, making an animal feed product similar to corn gluten feed. Example 9 depicts a prophetic example where corn fiber wet with water is extracted in ethanol and water, and where the extracted corn fiber is blended with steep liquor to make an animal feed product.

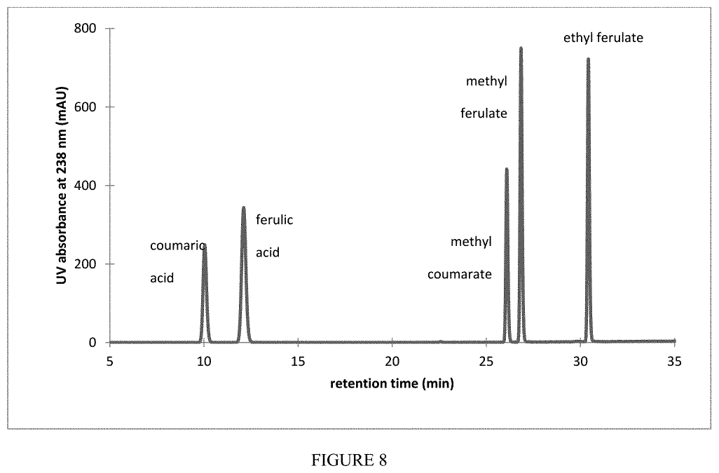

The resulting products in the liquid stream can be characterized by testing. For example, the liquid product stream can be analyzed with a high performance liquid chromatography (HPLC) equipped with a UV-vis detection. For example, a Zorbax SB-Phenyl reversed-phase C18 HPLC column can be used. A HPLC method can be used to quantify the mass of ferulate, coumarate, coumaric acid, and ferulic acid. A gradient of 1 mM of aqueous trifluoroacetic acid and acetonitrile can elute the products at 30° C. and a flow rate of 1.0 mL min-1. FIG. 8 shows a HPLC chromatogram for chemical standards of ferulic acid, coumaric acid, methyl ferulate, methyl coumarate, and ethyl ferulate. Standard curves for each ferulate and coumarate can be created to determine the concentration and yield of each product. The concentration of ferulate and ferulic acid can be determined at a wavelength of 326 or 238 nm while coumarate and coumaric acid can be quantified at 310 nm. Tests carried out as described herein on products from exemplary reactor runs was used to calculate the total yield (based on the starting mass of dry biomass) of ferulate, and the total yield from each biomass was near the theoretical content as shown in Table 1.

TABLE 1
Theoretical and extracted ferulate content of agricultural biomass

| | Theoretical ferulic acid | |
|---|---|---|
| | content | Extracted ferulate content |
| Biomass | (g_{ferulic acid}/g_{biomass}) | (g_{ferulate}/g_{biomass}) |
| Corn bran | 2.8-3.1 | 2.6 |
| Corn fiber | 1.0-1.8 | 1.6 |
| Corn gluten feed | 1.0-1.8 | 0.8 |
| Corn stover | | 0.4 |
| Miscanthus | 0.5-1.0 | 0.6 |

The resulting products in the liquid stream can be oils that can be characterized by testing. For example, the liquid product stream can be analyzed for oils with a HPLC equipped with a UV-vis detection. The column is a Zorbax SB-Phenyl reversed-phase C18 HPLC column. A HPLC method can be used to quantify the mass of oleate, linoleate, oleic acid, and linoleic acid, which comprised the majority of oils typically extracted from some biomass. An isocratic method using acetonitrile can elute the products at 30° C. and a flow rate of 1.0 mL min-1. Standard curves for each oleate and linoleate can be created to determine the concentration and yield of each product. The concentration of each compound can be determined at a wavelength of 205 nm. As described in Example 8 the ethyl esters of oleic acid and linoleic acid were extracted from corn fiber in combined yields of 3.8% with respect to the initial mass of corn fiber used in an exemplary reactive extraction.

The next step can include purification and/or filtration. A number of processing steps can be carried out on the products in the liquid, ferulate-containing stream. The liquid product stream containing ferulate and coumarate dissolved in the extracting solvent can be purified using filtration methods, chromatographic methods, or combinations thereof.

In some embodiments, the liquid permeate, which contains ferulate and coumarate, from the de-slurrying step can be subjected to microfiltration to remove solid particles in the size range of 0.1-100 microns. Advantageously, the microfiltration filter is designed to remove solids with particle sizes larger than 1-10 microns. The microfiltration step may also be preceded by a concentration step wherein the concentration of dissolved matter in the liquid fraction is increased prior to a microfiltration step or any subsequent processing. Concentration may be achieved using any number of process designs, including but not limited to distillation, vacuum distillation, evaporation, or organic solvent nanofiltration. 0-99% of the liquid phase solvent can be removed in this concentration step, corresponding to a 1-100-fold concentration. Preferably, at least about 90% of the solvent is removed, corresponding to a 10-fold concentration. In some embodiments, 50 to 90 percent of the solvent is removed. Some of the concentration may be achieved by flash depressurization of the reactive extraction product stream before it is cooled. In some embodiments, water can be added to the concentrated extract to selectively precipitate some impurities prior to the microfiltration step. Impurities such as proteins and lignin may precipitate in the concentration and water-addition steps and can be advantageously removed in the microfiltration stage. The solids recovered by microfiltration may be dried and combined with the coarse solid fraction from the de-slurrying step. The microfiltration solids can optionally be washed with clean solvent and re-filtered to recover any entrained product before being mixed with the coarse fraction and/or dried.

In some embodiments, the liquid permeate from the microfiltration stage, which contains ferulate and coumarate, can be subjected to ultrafiltration by means of an organic or inorganic membrane. The membrane can also be a nanofiltration membrane. Said membrane can be, for example, ceramic or polymeric in nature. Said membrane used for carrying out said ultrafiltration step has a cut-off threshold, typically defined by those skilled in the art as being the size starting from which 90% of a molecule is entirely retained by the membrane, of between 300 g/mol and 300,000 g/mol. Advantageously, the cut-off threshold can be between 300 g/mol and 10,000 g/mol. Said ultrafiltration step can be advantageously carried out at a temperature below about 80° C. In some embodiments, the ultrafiltration can be carried out between 30-60° C., or at about 50° C. It can be carried out at a pH of between about 5 and 12. In some embodiments, the pH can be between 7 and 9. The ultrafiltration membrane retains organic molecules such as polysaccharides, lignin, and proteins present in the liquid product stream resulting from the reaction and/or extraction of agricultural biomass with an extracting solvent. The ultrafiltration membrane allows ferulate and coumarate present in the liquid product stream resulting from the reactive extraction of agricultural biomass with an extracting solvent to pass through as the “permeate.” In some instances the ultrafiltration membrane retains organic molecules including oils. Some oils, such as oleates and linoleates, are known to be permeable. Said ultrafiltration stage can be used to purify ferulate, or as a pretreatment for downstream purification processes such as chromatography.

In some embodiments, the feed to an ultrafiltration unit can be comprised of a ferulate-containing biomass extract in anhydrous ethanol. The ultrafiltration membrane can have a nominal molecular weight cutoff-threshold of between 300 and 5000. Membranes with a nominal molecular weight cutoff-threshold of between 300 and 1000 Da may also be referred to interchangeably as nanofiltration membranes in this disclosure. Both ultrafiltration and nanofiltration membranes may be used to purify ferulates or ferulic acid. In some embodiments, the membrane's nominal molecular weight cutoff-threshold is about 600 Da. As an example of a suitable membrane, the membrane can be an Evonik PuraMem S600. The filtration can happen with a transmembrane pressure of 20-60 bar, or 30 bar, and at a temperature of 20-60° C., or 50° C. In other embodiments, the membrane's nominal molecular weight cutoff-threshold is 5000 Da. The membrane can be a Permionics HFUF 5 kDa and the filtration can happen with a transmembrane pressure of 1-10 bar, or 5 bar, and at a temperature of 5-60° C., or 50° C. The membrane can also be a Solecta PESK and the filtration can happen with a transmembrane pressure of 1-10 bar, or 5 bar, and at a temperature of 5-55° C., or 55° C. Example 10 depicts ultrafiltration of a solution consisting of ethyl ferulate extracted from corn fiber using a Permionics membrane. Example 11 depicts purification of ethyl ferulate from a corn fiber extract using an Evonik PuraMem S600 nanofiltration membrane. Example 12 portrays the subsequent removal of solvent by nanofiltration from an extract containing ethyl ferulate that was purified by ultrafiltration. Nanofiltration can be used to concentrate solutions of ferulate by selectively permeating solvent, while retaining ferulate and other dissolved components. Nanofiltration membranes can have a nominal molecular weight cutoff-threshold of less then 500 Daltons when used for concentration of solutions of ferulates. Nanofiltration can occur anywhere in the process as a solvent recovery step and an alternative to other concentration methods, such as distillation.

If the reactive extraction of ferulate occurs in an aqueous solution, ferulic acid can be extracted from the biomass. The ferulic acid can then be optionally processed using a transesterification reaction. Transesterification of the ferulic acid to an ester can occur after the reactive extraction of ferulic acid, but before purification by simulated moving bed chromatography. In some embodiments, the transesterification may be used to produce an ester that can be more easily separated in the chromatographic separation steps. Transesterification can occur before or after any micro or ultrafiltration.

In some embodiments, ferulic acid can be transesterified to ethyl ferulate using ethanol and a base catalyst. Ferulic acid can be dissolved in ethanol with a ferulic acid concentration of 5-200 g/L. The base can be the same as the reactive extraction base, and can include sodium hydroxide. The base can be of concentration 0.01-1 N. The transesterification can occur in a suitable reactor with a reaction temperature of 15-80° C.

If the reactive extraction occurs in a solution where the solvent is a mixture of ethanol and water, a mixture of ethyl ferulate and ferulic acid can be extracted from the biomass. In some embodiments, the reactive extraction occurs in a solvent of about 87 wt % ethanol and 13 wt % water, and yields a mixture of ferulic acid and ethyl ferulate. Purification of the reaction mixture by selective protein precipitation can optionally be achieved by concentration of the product mixture or by addition of water to the product mixture. Addition of water to the product mixture or concentration of the product mixture can be used to selectively precipitate proteins without precipitating ferulic acid or ethyl ferulate.

If the reactive extraction occurs in a solution where the solvent is a mixture of ethanol and water, a mixture of ethyl ferulate and ferulic acid can be extracted from the biomass. The ethyl ferulate can optionally be hydrolyzed to ferulic acid, increasing the total concentration of ferulic acid in the extraction product. In some embodiments, some or all of the ethanol in the ethyl ferulate and ferulic acid containing extraction product can be removed by distilling ethanol, nanofiltration, or other methods to remove ethanol. Partial or complete removal of ethanol from the reaction product maintains a mixture of ethyl ferulate and ferulic acid in a water rich solvent. To the water rich solution of ethyl ferulate and ferulic acid, a base including but not limited to sodium hydroxide can be added to induce hydrolysis of ethyl ferulate to ferulic acid. In some embodiments the water rich solution of ethyl ferulate, ferulic acid, and sodium hydroxide or other base is heated to accelerate hydrolysis of ethyl ferulate to ferulic acid. In some embodiments the mixture is heated to 20-80° C., or 35-55° C. In some embodiments, oils including but not limited to esters of oleic acid, oleic acid, esters of linoleic acid, and oleic acid can optionally be removed from the extraction product containing ferulic acid and ethyl ferulate prior to hydrolysis of ethyl ferulate to ferulic acid. In other embodiments the extraction product of ethyl ferulate and ferulic acid in a water and ethanol mixture can be made water rich by addition of water, rather than removal of ethanol prior to hydrolysis of ethyl ferulate to ferulic acid.

The next step can include the purification process. In the art of chromatography, the underlying physical principles driving separation of materials in a chromatographic process are the same whether separation is effected in a stationary column, true moving bed, simulated moving bed, or any other configuration columns or processes. It shall be understood from the outset that the chromatographic process laid out herein can be carried out in any combination of batch or semi-batch stationary chromatography systems, simulated moving bed, sequential simulated moving bed or any other mode of chromatographic separation. Similarly, the stationary adsorbents, eluents, and operating conditions listed herein are exemplary and are not intended to limit the choice of operating parameters. The scope and spirit of this disclosure shall therefore not be limited by the chromatographic processes illustrated herein below.

In a stationary bed chromatographic system, a mixture whose components are to be separated percolates through a container. The container can be cylindrical, and can be referred to herein as the column. The column contains a packing of a porous material (generally called the stationary phase) exhibiting a high permeability to fluids. The percolation velocity of each component of the mixture depends on the physical properties of that component so that the components exit from the column successively and selectively. Thus, some of the components tend to fix strongly to the stationary phase and thus will percolate slowly, whereas others tend to fix weakly and exit from the column more quickly. Many different stationary bed chromatographic systems have been proposed and are used for both analytical and industrial production purposes.

Simulated and moving bed chromatography (SMBC) is a known technique, familiar to those of skill in the art. The principle of operation involves countercurrent movement of a liquid eluent phase and a solid adsorbent phase. This operation allows minimal usage of solvent making the process economically viable. Such separation technology has found several applications in diverse areas, including hydrocarbons, industrial chemicals, oils, sugars and APIs.

A simulated moving bed system consists of a number of individual columns containing adsorbent which are connected together in series. Eluent is passed through the columns in a first direction. The injection points of the feedstock and the eluent, and the separated component collection points in the system, are periodically shifted by means of a series of valves. The overall effect is to simulate the operation of a single column containing a moving bed of the solid adsorbent. Thus, a simulated moving bed system consists of columns which, as in a conventional stationary bed system, contain stationary beds of solid adsorbent through which eluent is passed, but in a simulated moving bed system the operation is such as to simulate a continuous countercurrent moving bed.

The step time, i.e. the time between shifting the points of injection of the feed mixture and eluent, and the various take off points of the collected fractions, is not particularly limited, and will depend on the number and dimensions of the columns used, and the flow rate through the apparatus. A person of ordinary skill in the art would be able to determine appropriate step times to use in the process as disclosed herein.

Ethyl ferulate and ethyl coumarate may be isolated from each other during chromatographic separation and collected in separate fractions or streams, or they may be collected together in a single fraction or stream. It is further desirable that the process should involve inexpensive eluents which operate under standard temperature and pressure conditions.

The present disclosure therefore provides a chromatographic separation process for purifying the hydroxycinnamates ethyl ferulate and ethyl coumarate, from a feed mixture. In some embodiments, the chromatographic process comprises introducing the feed mixture to a simulated moving bed chromatography apparatus having a plurality of linked chromatography columns containing, as eluent, water or an aqueous base or aqueous acid or organic solvent or combination thereof, wherein the apparatus has a plurality of zones comprising at least a first zone and second zone, each zone having an extract stream and a raffinate stream from which liquid can be collected from said plurality of linked chromatography columns. The hydroxycinnamate products produced by the process of the present invention are produced in high yield, and have high purity.

Purification of ferulate and coumarate can be achieved using simulated moving bed chromatography alone or in combination with stationary chromatography systems and other chromatographic technologies including but not limited to normal-phase chromatography, reversed-phase chromatography, hydrophobic interactions chromatography, ion exchange chromatography, size exclusion chromatography, gel permeation chromatography, or other forms of chromatography and combinations thereof. Chromatographic purification of the hydroxycinnamates can be achieved before or after the aforementioned processes of concentration, impurity precipitation, ultrafiltration, nanofiltration, or combinations thereof with chromatographic processes.

The term “raffinate”, in the context of simulated moving bed chromatography, refers to the stream of components that move more rapidly with the liquid eluent phase compared with the solid adsorbent phase. Thus, in normal-phase chromatography, the raffinate stream is typically enriched in more non-polar components and depleted of more polar components. In reverse-phase chromatography, a raffinate stream is typically enriched with more polar components and depleted of less polar components compared with a feed stream.

The term “extract,” in the context of simulated moving bed chromatography, refers to the stream of components that move more rapidly with the solid adsorbent phase compared with the liquid eluent phase. Thus, in normal-phase chromatography, an extract stream is typically enriched with more polar components and depleted of more non-polar components compared with a feed stream. In reverse-phase chromatography, an extract stream is typically enriched with more non-polar components and depleted of more polar components compared with a feed stream.

Reverse-phase chromatography can used to purify hydroxycinnamates from impurities. A nonpolar stationary phase is used to adsorb and retain more non-polar components that are collected in an extract stream, while more polar components are carried through the column by a polar eluent. Ethyl ferulate is more non-polar than most components in the feed stream, so it adsorbs strongly to the non-polar stationary phase and is thus collected in the extract stream. Very non-polar oils such as ethyl linoleate and ethyl oleate may also be present in the feed stream and are collected in the extract stream. More polar components of the feed stream, which are largely impurities, move more rapidly in the eluent phase and are collected in a raffinate stream.

Stationary phases for reverse-phase chromatography can be resins that are macroporous and are composed of polystyrene, polystyrene crosslinked with divinyl benzene, octadecylsilane-bonded silica, octylsilane-bonded silica, polyvinylpyrrolidone, polymethacrylate, or other polymeric adsorbent. Stationary phases can also be modified to give them ion-exchange or other functionality.

One familiar with the art of chromatographic process development knows that the eluent composition is determined by investigating the separation of components as they progress through a test bed of resin. The composition of eluent is altered for a prospective resin until a desired separation factor between components is achieved. Because the optimal solvent composition is dependent on the specific resin and composition of the biomass extract feed, the purification of ferulates by chromatography in this invention is not limited to a specific solvent composition or resin.

Eluents for reverse-phase chromatography consist predominantly of a polar solvent, typically water or mixtures of water with organic solvent. The polarity of the eluent can be adjusted by the addition of a more non-polar co-solvent, creating a solvent mixture of intermediate polarity. The more non-polar co-solvent can be any organic solvent including but not limited to acetone, ethyl acetate, methanol, ethanol, higher alcohols, or combinations thereof. Mixtures of ethanol and water are preferred.

In some embodiments, the eluent for reverse-phase chromatography can consist of ethanol and water in the range of 50-100% ethanol and 0-50% water by volume, or 50% ethanol and 50% water by volume, 70-90% ethanol and 30-10% water by volume.

Normal-phase chromatography can used to purify hydroxycinnamates from impurities. A polar stationary phase is used to adsorb and retain more polar components, which are largely impurities, that are collected in an extract stream, while more non-polar components are carried through the column by a non-polar eluent. Ethyl ferulate is more non-polar than most components in the feed stream, so it does not adsorb strongly to the non-polar stationary phase and is thus collected in the raffinate stream. Very non-polar oils such as ethyl linoleate and ethyl oleate may also be present in the feed stream and are collected in the raffinate stream.

Stationary phases for normal-phase chromatography can be bare silica, alumina, zeolite, celite, silicates, magnesium silicates, or other inorganic, polar adsorbents. The particle and pore sizes of stationary phase materials are advantageously selected to increase the selectivity of the separation.

Eluents for normal-phase chromatography consist predominantly of a non-polar solvent, including but not limited to hexane, heptane, etc. or other non-polar solvents such as cyclohexane, benzene, toluene, diethyl ether, or mixtures thereof and mixtures of these solvents with more polar co-solvents. The polarity of the eluent can be adjusted by the addition of a more polar co-solvent, creating a solvent mixture of intermediate polarity. The more polar co-solvent can be water, or any organic solvent including but not limited to acetone, methyl ethyl ketone, ethyl acetate, methanol, ethanol, higher alcohols, or combinations thereof. Mixtures of hexanes and ethanol, or hexanes and ethyl acetate, are preferred.

In some operating modes, a regenerant solvent is used to desorb recalcitrant adsorbates from the stationary phase. In the case of reverse-phase chromatography, this regenerant can be ethanol, methanol, acetone, or any other organic solvent, as well as steam, and aqueous acids or bases. In the case of normal-phase chromatography, the regenerant can be water, ethanol, methanol, acetone, ethyl acetate, or any other organic solvent, as well as steam, and aqueous acids or bases that are compatible with the stationary phase.

A capture column is a packed bed that is used to remove certain components from a stream. Some components may adsorb too strongly to the stationary phase and be difficult to desorb in a continuous chromatographic process. When used in conjunction with a simulated moving bed process, a capture column placed before the simulated moving bed system can be used to remove impurities from a feed stream, capturing some of the total dissolved solids thereby preventing resin fouling. When used to treat the feed to a normal-phase chromatography system, the capture column can be a polar stationary phase of bare silica, alumina, zeolite, celite, silicates, magnesium silicates, or other inorganic, polar adsorbents.

Byproducts or coproducts besides hydroxycinnamates can be isolated from the biomass extract. The presence or specific ratios of coproducts is dependent on the reaction and/or extraction conditions and the biomass source. Coproducts include, but are not limited to: plant oils such as corn oil (typically comprised of oleate and linoleate esters or other fatty acids or fatty acid esters), polysaccharides such as B-glucans, proteins such as zeins, bio-phenols, and lignin oligomers. Recovery of these components is an optional step that can be achieved using simulated moving bed chromatography or other techniques such as precipitation, solvent extraction, or nanofiltration. For example, a second simulated moving bed chromatography system may be required to separate oils from the hydroxycinnamates.

Many SMBC operating modes could be used to achieve separation, including but not limited to 5-Zone, 2-Zone, I-SMB, Variable External Stream, 1-Column Analog, Varicol, ModiCon, Pseudo-SMB, SMB Cascade, Gradient-SMB, PowerFeed, SF-SMB, and Classical SMB chromatography systems. Multidirectional valves including rotary valves are used to switch streams. The simulated moving bed chromatography operating mode, column configuration, and valve choices are selected using methods that are obvious to a person skilled in the art.

In some embodiments, the SMBC operating mode is sequential simulated moving bed chromatography (SSMB).

In some embodiments, biomass extract containing the hydroxycinnamates ethyl ferulate and ethyl coumarate are purified using reverse-phase simulated moving bed (SMB) chromatography. The biomass extract, having been microfiltered then ultrafiltered to remove oligomeric lignin impurities and protein, can be contained in a stream of about 0-30% water in ethanol with a total dissolved solids content of about 0.5-100 g/L, about 10-50% of which is ethyl ferulate, which comprises the feed stream to the simulated moving bed system. In some embodiments, the stream can consist of a solvent of 0-50% water in ethanol, and have a total dissolved solids content of about 0.5-500 g/L. The feed stream is optionally microfiltered again to remove any precipitate or solids and is then fed to the simulated moving bed system. The eluent (mobile phase) can comprise 0-30% water in ethanol. The stationary phase used can be a macroporous resin comprised of polystyrene crosslinked with divinylbenzene. The average pore size of the resin can be between 40-300 Å, or between 50-100 Å. The average pore size of the resin can also be between about 10-1000 Å. The mesh particle size can be in the range 10-100 mesh, preferably 20-40 mesh. The mesh particle size can also be in the range of about 10-400 mesh. The SMB system can operate at ambient temperature or be heated to a temperature between about 20-80° C.

In some embodiments, and without wishing to be limited only to the resins named hereinafter, the chromatography stationary phase can be in the family of AMBERLITE resins, including AMBERLITE XAD-2, AMBERLITE XAD-4, AMBERLITE XAD-16, AMBERLITE FPX62, AMBERLITE FPX66, AMBERLITE FPX68. The chromatography stationary phase can also be in the family of DOWEX, including DOWEX Optipore L493. The chromatography stationary phase can also be in the AMBERCHROM family, including AMBERCHROM CG161M and AMBERCHROM CG300M. The chromatography stationary phase can also be in the SEPABEADS family, including SEPABEADS SP825L, SEPABEADS SP850, SEPABEADS SP70, AND SEPABEADS SP700. The chromatography stationary phase can also be in the PuroSorb family, including PuroSorb PAD350, PuroSorb PAD400, PuroSorb PAD550, PuroSorb PAD600, PuroSorb PAD900, and PuroSorb PAD1200. The chromatography stationary phase can also be in the Macronet family, including Macronet MN200, Macronet MN202, Macronet MN250, and Macronet MN270. The chromatography stationary phase can also be in the Chromalite family, including Chromalite 10AD1, Chromalite 10AD2, Chromalite 10MN, Chromalite 15AD1, Chromalite 15AD2, Chromalite 15MN, Chromalite PCG1200F15, Chromalite PCG600F, Chromalite PCG900F, Chromalite PCG1200F, Chromalite 70MN, Chromalite PCG600M, Chromalite PCG900M, Chromalite PCG1200M, Chromalite PCG600C, Chromalite PCG900C, and Chromalite PCG1200C. Example 13 describes the separation of ethyl ferulate from impurity components contained in a feed of ultrafiltered and de-oiled extract in an Agilent PLRP-S HPLC column, while Example 14 depicts the separation of an ultrafiltered and de-oiled extract feed in a single column of Chromalite PCG1200M.

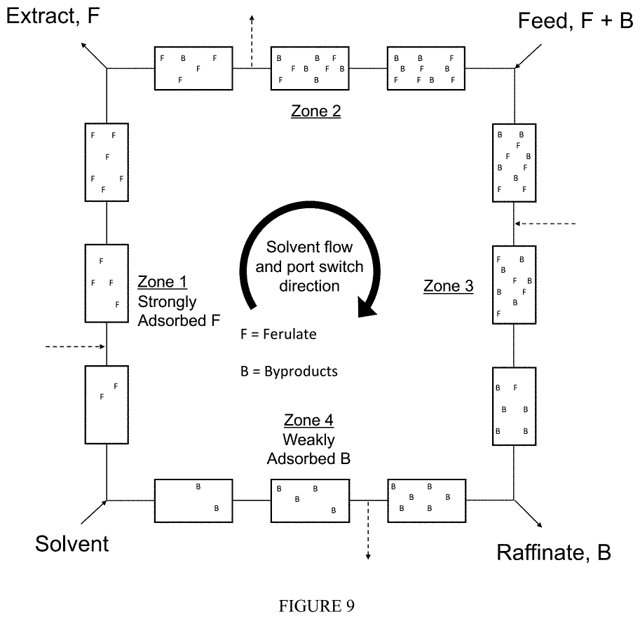

As the ferulate-containing feed enters the feed port and interacts with media, oils and ethyl cinnamates are more strongly adsorbed than impurity components in the feed. Eluent is pumped through the columns, moving the feed components through the column in the direction of eluent flow. Impurities flow through the columns faster than the ferulate and oil products, effecting separation between the two fractions. In cyclic steady-state operation, the impurities are collected downstream in a raffinate stream and the ferulate and oil products are collected in an extract stream located upstream from the current feed stream location. After a period of time, known to those skilled in the art as the port switching time, has passed, the valve configuration is changed to move the feed, eluent, extract, and raffinate streams to the next location. FIG. 9 shows a 4-zone, 3-3-3-3 column, simulated moving bed chromatography system that could be used to purify ferulates. The dashed lines in FIG. 9 represent the stream locations after the port switching time has passed. After time t*, the location of the inlet and outlet flows switch to the next port.

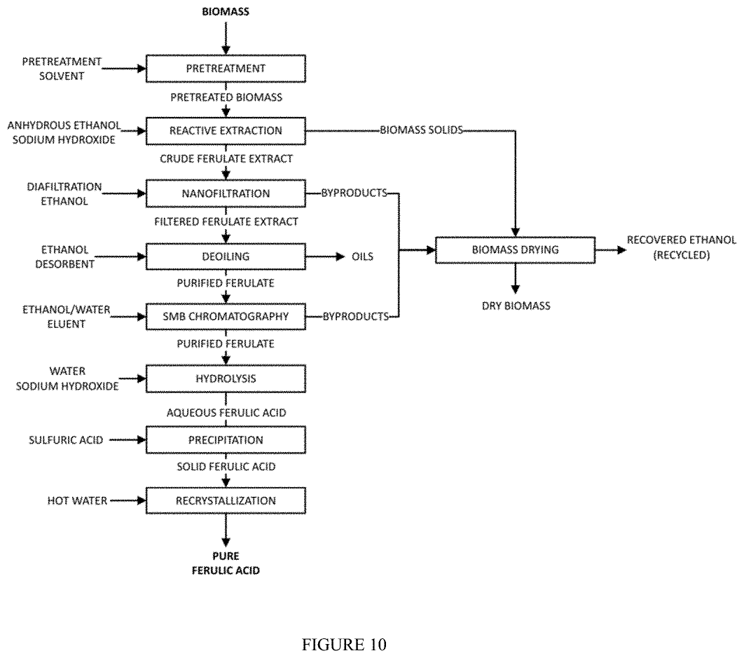

FIG. 10 depicts an example of an integrated process to produce ferulic acid from biomass using simulated moving bed chromatography as a purification. In a process similar to that depicted in FIG. 10, ethyl ferulate can be extracted from the biomass using anhydrous ethanol and after subsequent removal of the biomass solids, nanofiltration, and deoiling, the ethyl ferulate can be purified using simulated moving bed chromatography. Purified ethyl ferulate can then be hydrolyzed to ferulic acid and recovered as a solid, then recrystallized.

In some embodiments, a separate desorption cycle is used to strip recalcitrant oil from the column. Lignin and other more polar impurities are collected in the raffinate, ferulates with or without coumarates are collected in the extract stream, and recalcitrant oil (ethyl oleate and ethyl linoleate, typically) adsorbs most strongly. The column containing oil is periodically isolated from the active SMB system and is subjected to a separate oil desorption step. In this oil desorption step, a more non-polar desorbent is fed to the column. This desorbent can be ethanol, ethyl acetate, hexanes, or combinations thereof. In some embodiments, the desorbent can be acetone, any other organic solvent, steam, or acids or bases as solutions in water and/or organic solvent. The desorption step can also occur at a higher temperature than the SMB process. The temperature of the bed can be increased to desorb oil more quickly. After the oil is stripped from the column, the column is returned to the SMB operating temperature and any desorbent flushed with SMB eluent.

In some embodiments, recalcitrant oils or other aliphatic substances are instead removed prior to the main chromatography system. This de-oiling step can occur in a number of ways, including by adsorption of oils from the ferulate-containing solution and onto a stationary phase. De-oiling can occur at any point after extraction of ferulate from biomass, including before ultrafiltration or nanofiltration, and before chromatography or any subsequent purification. The feed to a de-oiling process is comprised of a ferulate-containing stream and aliphatic oils, which include oleic acid, linoleic acid, esters of oleic acid and linoleic acid, and any other aliphatic compounds. The composition of the de-oiling feed can be advantageously modified by adding water or solvent to the feed. In some embodiments, the biomass extract to be de-oiled is comprised of 96-99.5% ethanol by volume and water is added to the de-oiling feed until the solvent is comprised of 50-95% ethanol by volume, or 90% ethanol by volume. In other embodiments, the de-oiling feed is an aqueous solution. The stationary adsorbent is contained in one or more columns in parallel, in series, lead-lag series, lead-guard-regeneration, or in any other combination of columns or operating modes. Various operating adsorption modes can be utilized for the separation specific to the feed solution. A bed volume is the volume of resin contained in the column at the start of the de-oiling process.

The de-oiling can occur in a cyclic process. The column starts off in clean solvent with the same composition as the feed. If the feed is comprised of 90% ethanol by volume, the column can start in clean 90% ethanol by volume. In a loading step, liquid feed containing ferulate can be percolated through the adsorbent bed until at least one bed volume of feed has been loaded. The amount of feed loaded depends on the amount of oil in the feed and can vary with different biomass and extraction conditions. In some embodiments, 1 to 10 bed volumes of feed can be processed, although more bed volumes can be processed. The feed can be percolated through the adsorbent at a rate of 1-20 bed volumes per hour, or 1-6 bed volumes per hour. The composition of the column effluent can be analyzed for breakthrough of aliphatic compounds or oils to determine when the loading step is complete. The effluent, which contains ferulate and having been de-oiled, can be collected into a ferulate fraction.

Upon completion of loading, ferulate that remains in any residual liquid in the column can be recovered with a flushing step. The flushing step can use a flushing solvent that is the same composition as the ferulate-containing feed stream, which can be, for example, 90% ethanol and 10% water by volume or another solvent. At the same time, the ferulate-containing feed stream can be switched to a different column. In the flushing step, flushing solvent can be percolated through the bed. In some embodiments, 0 to 10 bed volumes of flushing solvent can be percolated through the bed, although any number of bed volumes can be used. The flushing solvent can be percolated through the adsorbent at a rate of 1-20 bed volumes per hour, or 1-6 bed volumes per hour. Recovered ferulate can be collected separately, or along with the remainder of the de-oiled feed as an aggregate ferulate fraction.

Upon completion of flushing, oils or aliphatic compounds adsorbed to the column can be removed in a desorption step. The desorption step can use a desorption solvent that can be comprised of a stronger organic solvent, which can be 100% ethanol, ethyl acetate, other organic solvents, steam, acids, bases, or any combination thereof. In the desorption step, desorption solvent can be percolated through the bed. In some embodiments, 1 to 10 bed volumes of desorption solvent can be percolated through the bed, although any number of bed volumes can be used. The desorption solvent can be percolated through the adsorbent at a rate of 1-20 bed volumes per hour, or 1-6 bed volumes per hour. The recovered oils can be collected as a separate oil fraction.

Upon completion of desorption, desorption solvent remains in the bed. To begin the next cycle of feed loading, the desorption solvent can be displaced in a desorbent displacement step. The desorbent displacement step can use a desorbent displacement solvent that can be the same composition as the ferulate-containing feed stream, which can be 90% ethanol and 10% water by volume or another solvent composition. In the desorbent displacement step, desorbent displacement solvent can be percolated through the bed. In some embodiments, 0 to 10 bed volumes of desorbent displacement solvent can be percolated through the bed, although any number of bed volumes can be used. The desorbent displacement solvent can be percolated through the adsorbent at a rate of 1-20 bed volumes per hour, or 1-6 bed volumes per hour. Desorbent solvent and any desorbent displacement solvent can be collected separately, or along with the remainder of the oil fraction as an aggregate oil fraction.

Deoiling can occur at ambient temperatures, or at other temperatures. Different steps in the cyclic de-oiling process can occur at different temperatures as well. Ferulate feed loading and flushing can occur at a temperature between about 5-60° C. Desorption of the oils is more effective at elevated temperatures and can advantageously occur at a higher temperature than the loading or flushing. Desorption of the oils can occur at a temperature between about 20-120° C., or at higher temperatures, or at the same temperature as loading or flushing. Example 15 depicts one cycle of a de-oiling process to remove corn oils from a corn fiber extract.

The next step in the process can comprise hydrolysis, precipitation, ion exchange, and/or other purification processes. The purified ferulate product can be subjected to hydrolysis after any of the prior purification steps. Any residual organic solvents or alcohols from prior purification steps can be removed from the liquid solution, leaving a solid or semi-solid substance containing ferulate. The semi-solid or solid can be dissolved in water with base, resulting in a basic aqueous solution containing ferulate of concentration of between 1-50 g/L, or between 10-20 g/L. The aqueous ferulate can be hydrolyzed under basic aqueous conditions to convert at least a portion of the ferulate to ferulic acid. In this process, additional base can be added to the ferulate solution and the solution can then be heated above about 20° C. to initialize the hydrolysis. Bases include any first or second group hydroxides such as sodium hydroxide or potassium hydroxide, carbonates, bicarbonates, ammonium, and any combination thereof. The base used can be the same or different than the base used in the initial reaction step with the biomass. In some embodiments, sodium hydroxide can be used as the base, and the sodium hydroxide can be used in concentrations between about 0.1-10 N, or between about 0.25-0.5 N. The mixture can be heated to between about 30-100° C. for about 0.1-5 hours. Under these conditions, ethyl- and methyl ferulate can hydrolyze (e.g., partially hydrolyze, substantially completely hydrolyze, or completely hydrolyze) to ferulic acid. In some embodiments, the mixture can also be heated to 40° C. in 0.25 N NaOH for 120 minutes.

In some embodiments, the resulting mixture containing the ferulic acid can be treated to precipitate byproducts of the reactive extraction process such as any lignin. The precipitation process can be carried out by acidifying the ferulic acid containing solution to selectively precipitate lignin and polysaccharides while the ferulic acid remains in solution. The concentration of ferulic acid can be adjusted to between about 1-20 g/L by addition of water to the ferulic acid containing solution, and the solution can be maintained at a temperature of between about 20-100° C. The ferulic acid solution can then be acidified to a pH of between about 3-6 by addition of an acid. Suitable acids useful in the acidification of the ferulic acid solution can include, but are not limited to, hydrochloric acid, nitric acid, phosphoric acid, sulfuric acid, acetic acid, and combinations thereof. In some embodiments the acid can be sulfuric acid. Upon acidification, the by-products can precipitate in the acidified solution. The acidified solution can be filtered to remove the precipitated byproducts including lignin and polysaccharides which precipitate as solid materials. In some embodiments, a filtration aid such as diatomaceous earth, celite, alumina, and/or silica may be added to the ferulic acid solution before acidification. Upon acidification, precipitated byproducts such as lignin and polysaccharides can bind to the inert filtration aid, increasing the ease of filtration and preventing fouling. In some embodiments, centrifugation can be used to isolate the solid precipitated lignin and polysaccharides from the acidified solution in place of filtration.

In some embodiments, oily materials may form a distinct phase on top of the aqueous phase before or after acidification. These oily materials can be removed from the aqueous phase using liquid/liquid phase separation, including decanting.

Aqueous solutions of ferulic acid resulting from hydrolysis can be further purified using a variety of processing steps. The aqueous solution containing ferulic acid can be further acidified to precipitate ferulic acid. In some embodiments, ferulic acid is precipitated after impurities have been precipitated using acid treatment. In other embodiments, ferulic acid is precipitated directly from the hydrolysis product solution. The ferulic acid solution can then be acidified to a pH of between about 1-3 by addition of an acid. Suitable acids useful in the precipitation of ferulic acid solution can include, but are not limited to, hydrochloric acid, nitric acid, phosphoric acid, sulfuric acid, acetic acid, and combinations thereof. In some embodiments the acid can be sulfuric acid. Upon acidification, the ferulic acid can precipitate in the acidified solution. The acidified solution can be cooled to a temperature of between about 2-30° C. to reduce the solubility of ferulic acid and aid in the precipitation process. Crude crystalline ferulic acid solids can be recovered from the solution using filtration. In some embodiments, the crude crystalline ferulic acid is washed with 2-30° C. of water with mass 1-100 times the recovered crystals. In Example 16, ferulate is hydrolyzed to ferulic acid, which is precipitated from solution using acid. In Example 17, the ethyl ferulate product of reverse phase chromatography is hydrolyzed and precipitated.

In some embodiments, the aqueous solution of ferulic acid resulting from the hydrolysis step can be further purified using a liquid-liquid extraction process using an organic solvent. In this step of a purification process, the liquid-liquid extraction can be used to extract ferulic acid from an aqueous solution to an organic solvent. Suitable organic solvents may include, but are not limited to, ethyl acetate, diethyl ether, dichloromethane, hexane, heptane, pentane, toluene, xylenes, and mixtures thereof. In some embodiments, the organic solvent can be or include ethyl acetate. The aqueous solution containing ferulic acid can be further acidified to a pH of between about 1-5 by addition of an acid before liquid-liquid extraction with an organic solvent. Suitable acids can include, but are not limited to, hydrochloric acid, nitric acid, phosphoric acid, sulfuric acid, acetic acid, and combinations thereof. In some embodiments, the acid can be or include sulfuric acid. Upon acidification, the aqueous ferulic acid solution can be extracted with the organic solvent using liquid-liquid contact. Typically, the volume of organic solvent used to extract the ferulic acid is between about 0.5-3 times the volume of the aqueous ferulic acid solution. Following liquid-liquid extraction of the ferulic acid into the organic solvent, the organic solvent phase can be separated from the aqueous phase, and the organic solvent can then be removed by evaporation (e.g., rotary evaporation, etc.) to yield solid or semi-solid ferulic acid. In some embodiments, the solid or semi-solid ferulic acid can have a purity of between about 10-50% or between about 20-40% by mass. The resulting ferulic acid can be used as product or subjected to further purification depending on the use and product purity requirements.

The present disclosure includes a method of purifying ferulic acid, which is a carboxylic acid from a solution containing the ferulic acid comprising the steps of: (a) applying the solution to an anionic exchange resin; (b) washing the resin under conditions that cause removal of neutral molecules, cations, large molecular weight compounds, and other biomass extractives and allow retention of carboxylic anions on the resin; (c) displacing the carboxylic acid from the resin by washing the resin with an amount of a stronger anion effective to displace substantially all of the carboxylic acid; and (d) recovering the carboxylic acid in the eluent. In an embodiment, the method of the present invention comprises regenerating the resin after the carboxylic acid displacement step by treating the resin with a strong base in an amount sufficient to allow substantial exchange of inorganic anion bound to the resin with hydroxide ions.

In an embodiment, the proposed operation allows the anion exchange resin to be recycled. The proposed exchange process is as follows. First, the ferulic acid can be adsorbed to a strong or weak anion exchange resin by the formation of the ionic bonds between the carboxylic acid molecule and exchange sites on the resin. At this stage, most of the neutral or cationic material, or large molecules or cellular debris, pass through the resin. Next, the resin is washed with a liquid such as water to remove unbound contaminants that are trapped in the ion exchange resins. Following the wash step, ferulic acid is displaced from the resin by exchanging the adsorbed carboxylic ion for a stronger, inorganic ion, thereby causing the release of ferulic acid. The anion exchange resin is prepared for further cycles of ferulic acid adsorption by regenerating the anion exchange sites on the resin. This is accomplished by treating the resin with a strong base, including but not limited to sodium hydroxide, which causes the inorganic anion to be exchanged for a hydroxide ion. The net result is an acidified, purified ferulic acid product.

Anion exchange resins exist in salt or base forms. In order to adsorb the organic anion from organic salt mixture, the resin must be converted from the salt form to the base form during the regeneration procedure with sodium hydroxide. Without wishing to be limited by theory, the typical steps of the ion exchange purification process are as follows:

1) Regeneration of resin exchange sites

R_{z}.Cl+NaOH⇄R_{z}.OH+NaCl
(R_{z}=Resin)

2) Adsorption of carboxylate anion to resin

R_{z}OH+X—R_{c}⇄R_{z}.R_{c}+X.OH
(R_{c}=Ferulic acid ion; X—=Inorganic/proton)

3) Displacement and release of carboxylic ion as carboxylic acid

R_{z}.R_{c}+HR_{I}⇄R_{z}.R_{I}+HR_{c}
(HR_{I}=Inorganic acid; HR_{c}=Ferulic acid)

4) Release of inorganic anion and regeneration of resin

R_{z}.R_{I}+NaOH⇄R_{z}.OH+NaR_{I}

In general, any strong or weak ion exchange resin, including Type I or II can be used for ion exchange purification of ferulic or coumaric acid, including those manufactured by Rohm and Haas, Dow, DuPont, Purolite, or others. One skilled in the art will appreciate that any strong base anion exchange resin could be used in the method of the present invention. Resin properties considered in the selection include the relative affinity of the resin for the targeted organic or amino acid ions versus other impurities, the capacity to adsorb carboxylic ions, and ability to release during displacement.

In some embodiments, water can be used during the wash step to remove unbound materials from the resin. However, any liquid that provides suitable wash conditions can be used in the wash step. Suitable wash conditions are conditions that allow removal of neutral or cationic molecules, large molecules, and other biomass extractives, while at the same time allowing most of the bound carboxylate anions to be retained on the resin. Examples of other suitable washes include certain buffer solutions and solvents, which can include ethanol or other organic solvents. A solution used as a wash in the present invention should have characteristics similar to water, in that it should be capable of removing substantially all unwanted materials, while allowing most of the anion to remain bound to the resin.

The release step can be conducted by using a strong inorganic acid at a concentration such that the number of equivalents applied to the column equals or, more preferably, exceeds the equivalents of carboxylate anion bound to the resin. The inorganic acid can be comprised of any suitable strong acids, including, but not limited to, hydrochloric acid, sulfuric acid, nitric acid, and phosphoric acid. The release step can use a co-solvent, which can be an organic solvent, to improve the solubility of the ferulic acid. The co-solvent can be ethanol, and comprise 0-100% of the solvent in addition to any acid or base.

In some embodiments, the release step can be conducted using a strong inorganic base, which can be sodium hydroxide or any other inorganic base. The base not only acts to solubilize the ferulic acid by deprotonation of the phenol and the carboxylic acid, but also to regenerate the resin in a single step. Ferulic acid can be precipitated directly from the solution of base by acidification with any suitable acid.

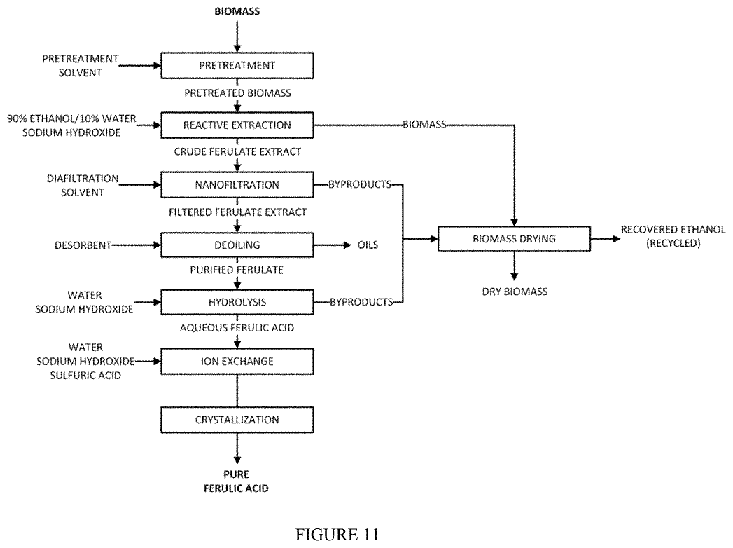

After the ferulic acid is removed from the resin, the product stream can be subjected to further purification, including crystallization such as evaporative crystallization, or recrystallization. Any suitable purification method for purifying the ferulic acid can be used in the present invention. FIG. 11 depicts an example of an integrated process to produce ferulic acid from biomass using ion exchange as a purification. In FIG. 11, the ethyl ferulate and ferulic acid can be extracted from the biomass using 90% ethanol. After subsequent removal of the biomass solids, nanofiltration, and deoiling, the ferulate can be hydrolyzed to ferulic acid, which can be purified using ion exchange. Example 18 prophetically describes a process wherein ferulate and ferulic acid are extracted from corn fiber using 90% ethanol and are hydrolyzed to ferulic acid, which is purified using ion exchange.

In addition to purifying ferulic acid, the method of the present invention can be used to produce the corresponding esters in pure form. With the proper selection of ion exchange resin, a ferulate anion could be first adsorbed and then eluted with an alcohol to produce corresponding ester in pure form. Factors to consider in selecting the resin include its stability in the solvent to be used, its stability at increased temperatures, and its ability to release the anion when treated with the selected solvent. The selected ion exchange resin and the inorganic acid could be used as the esterification catalysts also.

In some embodiments, and without wishing to be limited only to the ion exchange resins named hereinafter, the ion exchange resin can be Purolite A5100H, Purolite A103SPLUS, or any other anion exchange resin.

The solid or semi-solid ferulic acid can be further purified using a dissolution-precipitation or recrystallization process. In some embodiments, the solid ferulic acid of low purity (e.g., between about 20-90% purity by mass) can be further purified through a hot filtration and precipitation process. In this process, the solid ferulic acid can be dissolved in water at a concentration of 1-30 g/L ferulic acid and heated to between about 60-100° C. In some embodiments, the concentration of ferulic acid dissolved in the water can be 1-500 grams per liter of water. The heated mixture can be filtered to remove non-soluble materials while maintaining a temperature of between about 60-100° C. The filtrate can then be cooled to precipitate solid ferulic acid at a purity greater than the starting purity. In some embodiments, the dissolution-precipitation process can increase the purity of the solid ferulic acid to between about 60-99% purity by mass. Alternatively, the concentration of ferulic acid in the filtrate can be increased from 1-10 g/L to 10-35 g/L by evaporating a portion of the aqueous filtrate prior to cooling to precipitate solid ferulic acid with a purity of between about 80-99% by mass. The dissolution-precipitation recrystallization process can be repeated several times to reach higher purities as necessary. In Example 19, a holistic process to produce ferulic acid from a corn fiber extract is depicted, wherein the ferulic acid is recrystallized near the end of the process.

### EXAMPLES

The embodiments having been generally described, the following examples are given as particular embodiments of the disclosure and to demonstrate the practice and advantages thereof. It is understood that the examples are given by way of illustration and are not intended to limit the specification or the claims in any manner.

### Example 1

Pretreatment and reactive extraction of ethyl ferulate and ethyl coumarate from corn fiber. In this example, 225 g of corn fiber that was previously dried at 90° C. under vacuum for 50 hr was loaded into a 7.5 L stirred batch reactor (Parr Instruments 4550) with 3.6 L of deionized water. The reactor was sealed and purged with nitrogen (Airgas NI 300) three times by pressurizing the reactor to ca. 6 bar and subsequently venting the pressure to ca. 1 bar. The temperature was increased to 80° C. at a ramp rate of 220° C.^{hr-1} while the reactor was stirred at 200 rpm. The temperature was held at 80° C. for 2 hours before returning to room temperature. After the pretreatment, the water solvent and solids were filtered through a 75 μm mesh filter bag and rinsed with 1 L of deionized water. The pretreated fiber was then dried in an oven at 80° C. for 4 days. The recovered 164.8 g of dried, pretreated fiber was then loaded into a 7.5 L stirred batch reactor (Parr Instruments 4550) with 3.6 L of 0.04 M sodium hydroxide in 200 proof anhydrous ethanol. The reactor was sealed and purged with nitrogen (Airgas NI 300) three times by pressurizing the reactor to ca. 6 bar and subsequently venting the pressure to ca. 1 bar. The temperature was increased to 145° C. at a ramp rate of 220° C.^{hr-1} while the reactor was stirred at 200 rpm. The temperature was held at 145° C. for 4 hours before returning to room temperature. After the reaction, the ethanol solvent and solids were filtered through a 75 μm mesh filter bag and rinsed with 1 L of ethanol. Fine solids were removed from the extract by filtering through filter paper (particle retention >11 μm) and rinsed with 25 mL ethanol. The yield of ethyl ferulate in the filtrate was analyzed by HPLC. The yield of ethyl ferulate was determined to be 1.7% with respect to the 225 g of corn fiber subjected to pretreatment. The purity was determined from the yield of ethyl ferulate and the total dissolved solids in the extract. The purity of ethyl ferulate in the extract was 12.6% by mass.

### Example 2

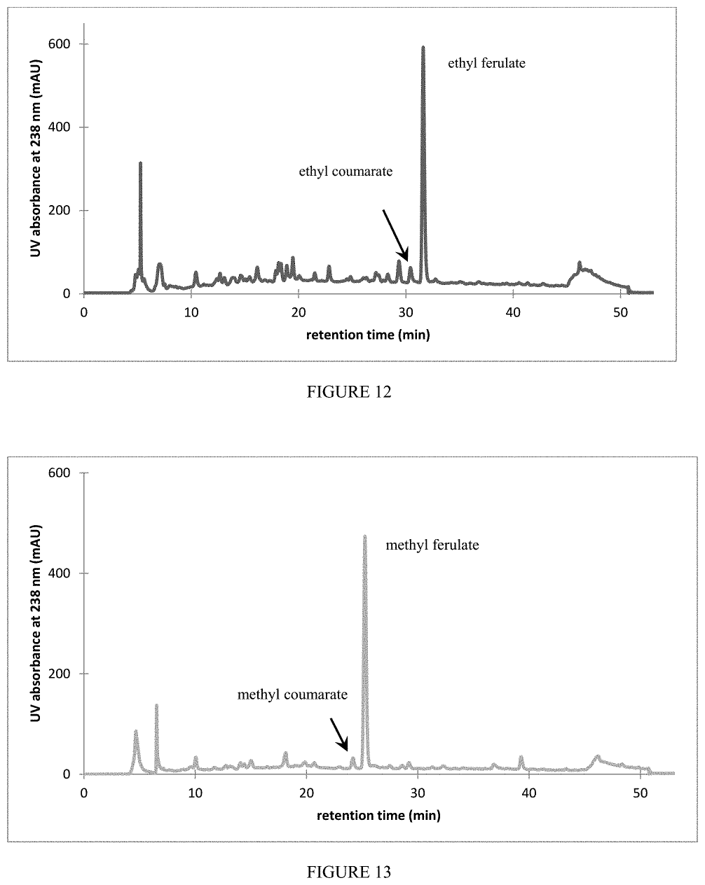

Reactive extraction of ethyl ferulate from corn fiber. In this example, 222 g of corn fiber that was previously dried at 90° C. under vacuum for 50 h was loaded into a 7.5 stirred batch reactor (Parr Instruments 4550) with 3.6 L of 0.04 M sodium hydroxide in 200 proof anhydrous ethanol. The reactor was sealed and purged with nitrogen (Airgas NI 300) three times by pressurizing the reactor to ca. 6 bar and subsequently venting the pressure to ca. 1 bar. The temperature was increased to 145° C. at a ramp rate of 220° C.^{hr-1} while the reactor was stirred at 200 rpm. The temperature was held at 145° C. for 4 hours before returning to room temperature. After the reaction, the ethanol solvent and solids were filtered through a 75 μm mesh filter bag and rinsed with 1 L of ethanol. Fine solids were removed from the extract by filtering through filter paper (particle retention >11 μm) and rinsed with 25 mL ethanol. The yield of ethyl ferulate in the filtrate was analyzed by HPLC and the chromatogram shown in FIG. 12 was generated. The yield of ethyl ferulate was determined to be 1.6% with respect to the 222 g of initial corn fiber.

### Example 3

Reactive extraction of methyl ferulate and methyl coumarate from a continuous reactor. In this example, 172.3 g of corn fiber previously dried at 100° C. for 24 hr was loaded into a 1 L packed bed reactor which was insulated inside a furnace. The back pressure regulator was set to 7 bar, and the reactor was filled with methanol at a flow rate of 3 mL min⁻¹. The back pressure regulator was increased to 46.9 bar while the furnace temperature was ramped such that an internal thermocouple in contact with the corn fiber at the exit of the packed bed recorded a temperature of 200° C., which required 3 hr. The packed bed was maintained at 46.9 bar and 200° C. for 6 hr before the furnace was turned off and the methanol flow rate was increased to 10 mL min⁻¹ for 10 min to flush the corn fiber bed. The methanol flow was stopped and the reactor was cooled to room temperature overnight. Methanol from the system was collected in a reservoir with a total volume of 1.81 L. The methyl ferulate content of the oil was analyzed by HPLC, as shown in FIG. 13. Methyl coumarate and methyl ferulate eluted at ca. 24.2 and 25.3 min, respectively. The yield of methyl ferulate and methyl coumarate was determined to be 1.5% and 1.3%, respectively, with respect to the total dry biomass (assuming 8% moisture content of the biomass, dry mass=0.92*172.3 g=158.5 g). The hourly extraction of methyl ferulate and methyl coumarate is shown in FIG. 14. As shown in FIG. 14, time 0 hr corresponds to the first time the internal bed temperature at the exit was 200° C.

### Example 4

Reactive extraction of ethyl ferulate from corn fiber with varying moisture levels. In this example, a single batch of corn fiber was divided into two portions (A and B) and dried in an oven at 120° C. Corn fiber (A) was dried until the corn fiber contained 12.3% water by mass. Corn fiber (B) was dried until the corn fiber contained 4% water by mass. The dried corn fiber samples A and B were then extracted in separate reactive extractions. In the first extraction, 450 g corn fiber A containing 12.3% water by mass was loaded into a 7.5 stirred batch reactor (Parr Instruments 4550) with 3.6 L of 0.12 M sodium hydroxide in 200 proof anhydrous ethanol. The ratio of solvent to biomass was 6.3:1 by mass. The reactor was sealed and purged with nitrogen (Airgas NI 300) three times by pressurizing the reactor to ca. 6 bar and subsequently venting the pressure to ca. 1 bar. The temperature was increased to 145° C. at a ramp rate of 4° C. per minute while the reactor was stirred at 200 rpm. Following the 4 hour soak at 145° C., the reaction was cooled to room temperature. After the reaction, the ethanol solvent and solids were filtered through a 75 μm mesh filter bag and rinsed with 1 L of ethanol. Fine solids were removed from the extract by filtering through filter paper (particle retention >11 μm) and rinsed with 25 mL ethanol. The yield of ethyl ferulate and ethyl coumarate in the filtrate was analyzed by HPLC. The yield of ethyl ferulate was determined to be 4.72 g, 1.20% with respect to the 450 g of corn fiber loaded into the reactor which after subtraction of the 12.3% moisture had a dry mass of 394 g. In a similar fashion, 181 g of corn fiber B containing 4% water by mass was loaded into a 7.5 stirred batch reactor (Parr Instruments 4550) with 1.45 L of 0.12 M sodium hydroxide in 200 proof anhydrous ethanol. The ratio of solvent to biomass was 6.3:1. The reactor was sealed and purged with nitrogen (Airgas NI 300) three times by pressurizing the reactor to ca. 6 bar and subsequently venting the pressure to ca. 1 bar. The temperature was increased to 145° C. at a ramp rate of 4° C. per minute while the reactor was stirred at 200 rpm. Following the 4 hour soak at 145° C., the reaction was cooled to room temperature. After the reaction, the ethanol solvent and solids were filtered through a 75 μm mesh filter bag and rinsed with 500 mL of ethanol. Fine solids were removed from the extract by filtering through filter paper (particle retention >11 μm) and rinsed with 25 mL ethanol. The yield of ethyl ferulate and ethyl coumarate in the filtrate was analyzed by HPLC. The yield of ethyl ferulate was determined to be 3.05 g, 1.75% with respect to the 181 g of corn fiber loaded into the reactor which after subtraction of the 4% moisture had a dry mass of 174 g.

### Example 5

Reactive extraction of ethyl ferulate and ferulic acid mixture. In this example 225 g of corn fiber that was previously dried at 80-100° C. was loaded into a 7.5 stirred batch reactor (Parr Instruments 4550). To the reactor was added 360 g water containing 11.535 g sodium hydroxide. 2556 g of 200 proof anhydrous ethanol was then added to the reactor. The reactor was sealed and purged with nitrogen (Airgas NI 300) three times by pressurizing the reactor to ca. 6 bar and subsequently venting the pressure to ca. 1 bar. The temperature was increased to 120° C. at a ramp rate of 4° C. per minute while the reactor was stirred at 200 rpm. After the reaction mixture reached a temperature of 120° C. (29 minutes from beginning of temperature ramp), a 20 mL sample of the reaction mixture was removed using a sampling port on the reactor and HPLC analysis showed the yields of ferulic acid and ethyl ferulate to be 0.2% and 0.22%, respectively. The reaction temperature was maintained at 120° C. and another 20 mL sample of the reaction mixture was removed 38 minutes from the beginning of the temperature ramp and HPLC analysis showed the yields of ferulic acid and ethyl ferulate to be 0.4% and 0.47%, respectively. The reaction temperature was maintained at 120° C. and another 20 mL sample of the reaction mixture was removed 62 minutes from the beginning of the temperature ramp and HPLC analysis showed the yields of ferulic acid and ethyl ferulate to be 0.4% and 0.62%, respectively. The reaction temperature was maintained at 120° C. and another 20 mL sample of the reaction mixture was removed 180 minutes from the beginning of the temperature ramp and HPLC analysis showed the yields of ferulic acid and ethyl ferulate to be 0.1% and 0.70%, respectively. The yields of ferulic acid and ethyl ferulate were determined in each sample analyzed by with respect to the 225 g of initial corn fiber. FIG. 6 shows the yields of ethyl ferulate, ferulic acid, and combined total yield ferulic acid and ethyl ferulate plotted as a function of time for this reaction. As illustrated in FIG. 6, a maximum combined yield of ferulic acid and ethyl ferulate was observed at 1.15 hours after the start of the reaction and the combined yield of ferulic acid and ethyl ferulate decreased after 1.15 hours of reaction time until conclusion of the reaction at 2.8 hours.

### Example 6

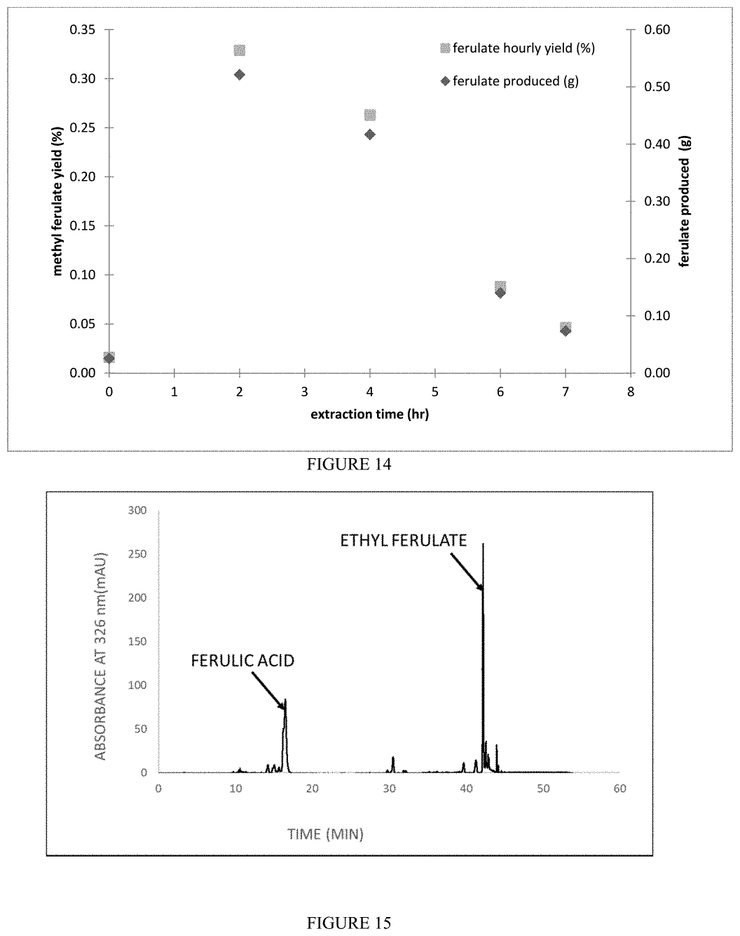

Reactive extraction of ethyl ferulate and ferulic acid mixture at 100° C. using 0.08 M sodium hydroxide. In this example 225 g of corn fiber that was previously dried at 80-100° C. was loaded into a 7.5 stirred batch reactor (Parr Instruments 4550). To the reactor was added 360 g water and 11.535 g sodium hydroxide. 2556 g of 200 proof anhydrous ethanol was then added to the reactor. The reactor was sealed and purged with nitrogen (Airgas NI 300) three times by pressurizing the reactor to ca. 6 bar and subsequently venting the pressure to ca. 1 bar. The temperature was increased to 100° C. at a ramp rate of 4° C. per minute while the reactor was stirred at 200 rpm. After the reaction mixture reached a temperature of 100° C., the reactor was sampled periodically throughout the 4 hour soak at 100° C., before cooling to room temperature. HPLC analysis was used to determine the yields of ferulic acid and ethyl ferulate throughout the course of the reaction. The yields of ferulic acid and ethyl ferulate were determined in each sample analyzed by HPLC with respect to the 225 g of initial corn fiber. FIG. 15 shows the HPLC chromatogram of the final reaction products to be ferulic acid and ethyl ferulate. FIG. 5 shows the yields of ethyl ferulate, ferulic acid, and combined total yield ferulic acid and ethyl ferulate plotted as a function of time over the duration of this reaction. As illustrated in FIG. 5, the combined yields of ferulic acid and ethyl ferulate continued to increase throughout the reaction. As shown in FIG. 5, the yield of ethyl ferulate was 0.66% with respect to the initial 450 g corn fiber, and the yield of ferulic acid was 0.45% with respect to the initial 45 g corn fiber.

### Example 7

Reactive extraction of ethyl ferulate and ferulic acid mixture at 100° C. using 0.16 M sodium hydroxide. In this example, 450 g of corn fiber that was previously dried at 80-100° C. was loaded into a 7.5 stirred batch reactor (Parr Instruments 4550). To the reactor was added 360 g water and 23 g sodium hydroxide. 2556 g of 200 proof anhydrous ethanol was then added to the reactor. The reactor was sealed and purged with nitrogen (Airgas NI 300) three times by pressurizing the reactor to ca. 6 bar and subsequently venting the pressure to ca. 1 bar. The temperature was increased to 100° C. at a ramp rate of 4° C. per minute while the reactor was stirred at 200 rpm. After the reaction mixture reached a temperature of 100° C., the reactor was sampled periodically throughout the 4 hour soak at 100° C., before cooling to room temperature. HPLC analysis was used to determine the yields of ferulic acid and ethyl ferulate throughout the course of the reaction. The yields of ferulic acid and ethyl ferulate were determined in each sample analyzed by HPLC with respect to the 450 g of initial corn fiber. FIG. 7 shows the yields of ethyl ferulate, ferulic acid, and combined total yield ferulic acid and ethyl ferulate plotted as a function of time for this reaction. As illustrated in FIG. 7, a maximum yield of 0.62% ethyl ferulate and 0.41% ferulic acid was observed after the reaction had been held at a temperature of 100° C. for 160 minutes.

### Example 8

Reactive extraction of ethyl ferulate from corn fiber with solvent flash and fiber washing. In this example, 450 g of corn fiber was first dried 120° C. until the moisture in the fiber was about 0.5% by mass. The dried corn fiber was then loaded into a 7.5 stirred batch reactor (Parr Instruments 4550) with 3.6 L of 0.12 M sodium hydroxide in 200 proof anhydrous ethanol. The reactor was sealed and purged with nitrogen (Airgas NI 300) three times by pressurizing the reactor to ca. 6 bar and subsequently venting the pressure to ca. 1 bar. The temperature was increased to 145° C. at a ramp rate of 240° C.^{hr-1} while the reactor was stirred at 200 rpm. The temperature was held at 145° C. for 4 hours. Following the 4 hour soak at 145° C., 1.15 L of solvent was evaporated from the reaction mixture by solvent flash evaporation and the reaction mixture was cooled to room temperature. The remaining ethanol solvent and solids were filtered through a 75 μm mesh filter bag, rinsed with 450 mL of ethanol, and pressed to remove solvent containing ethyl ferulate from the fiber. After pressing the solids, the solids contained in the filter bag were rinsed with an additional 450 mL ethanol and pressed for a second time to remove solvent containing ethyl ferulate from the fiber. The yield of ethyl ferulate was determined using HPLC analysis to be 6.96 g, 1.55% with respect to the 450 g of initial corn fiber. The yield of ethyl coumarate was 0.52 g, 0.12% with respect to the 450 g of initial corn fiber. The yield of corn oil was measured by HPLC analysis of the ethyl esters of oleic acid and linoleic acid. The combined yield of the ethyl esters of oleic acid and linoleic acid was 17.1 g, 3.8% with respect to the initial 450 g corn fiber used in the reaction.

### Example 9

Reactive extraction of ethyl ferulate and ferulic acid from wet corn fiber. In a prophetic example, wet corn fiber containing 50% by mass water was received from a corn milling facility. 818 g of wet corn fiber which contained 414 g water and 414 g bone dry corn fiber was added to a 7.5 stirred batch reactor (Parr Instruments 4550). To the reactor was added 3.726 L anhydrous ethanol. When considering the water contained in the corn fiber and the ethanol charged to the reactor, the total reaction solvent contained 87% by mass ethanol and 13% by mass water. 26.5 g Sodium hydroxide was added to the reactor to bring the total concentration of sodium hydroxide to 0.16 M. The reactor was sealed and purged with nitrogen (Airgas NI 300) three times by pressurizing the reactor to ca. 6 bar and subsequently venting the pressure to ca. 1 bar. The temperature was increased to 100° C. at a ramp rate of 240° C. h⁻¹ while the reactor was stirred at 200 rpm. The temperature was held at 100° C. for 4 hours. Following the 4 hour soak at 100° C., the reaction mixture was cooled to room temperature. The remaining ethanol solvent and solids were filtered through a 75 μm mesh filter bag, rinsed with 450 mL of ethanol, and pressed to remove solvent containing ethyl ferulate and ferulic acid from the fiber. After pressing the solids, the solids contained in the filter bag were rinsed with an additional 450 mL ethanol and pressed for a second time to remove solvent containing ethyl ferulate and ferulic acid from the corn fiber. After the remaining corn fiber had been pressed to remove ethyl ferulate and ferulic acid, a protein containing steep liquor which was produced from a wet corn milling process was added to the extracted corn fiber and dried to make an animal feed similar in nature to corn gluten feed.

### Example 10

Purification of ethyl ferulate and ethyl coumarate using membrane filtration. In this example, crude ethyl ferulate extract was purified using ultrafiltration. One circular Permionics polymeric, flat-sheet ultrafiltration membrane with a molecular weight cutoff of 5 kDa was loaded into one 4″ crossflow ultrafiltration cell in an Evonik METCell Crossflow filtration system. The membrane was conditioned by filling the filtration system with 640 mL of anhydrous ethanol, circulating the ethanol over the retentate side of the membrane at 1 L/min using a gear pump, and pressurizing the system with nitrogen gas to a pressure of 5 barg. The membrane permeated ethanol until 25 mL of ethanol was collected from the cell, at which point the system was depressurized and the conditioning ethanol was drained. 650 mL of the crude ethyl ferulate extract, which contained 8.15 g/L of total dissolved solids and 0.776 g/L of ethyl ferulate, was loaded into the filtration system. The extract was continuously circulated over the retentate side of the membranes at 1 L/min using a gear pump, then the system was pressurized to 5 barg using nitrogen gas. Ethanol containing ferulate permeated through the membrane, while some impurities such as proteins and lignin were retained by the membrane. 50 mL of permeate from the cell was discarded until pseudo-steady-state was reached, then permeate collection began. The average permeate flux was 20.5 L/(m² hr). The permeate was collected over one hour and pooled into an aggregate permeate sample. The aggregate permeate had a total dissolved solids content of 3.82 g/L, corresponding to a 53% reduction in total dissolved solids, and 0.678 g/L of ethyl ferulate, corresponding to an ethyl ferulate passage rate of 87%. The purity of ethyl ferulate increased from 10% in the feed to 18% in the permeate.

### Example 11

Purification of ethyl ferulate and ethyl coumarate using organic solvent nanofiltration. In this example, crude ethyl ferulate extract was purified using nanofiltration. Two circular, polymeric, flat-sheet Evonik PuraMem S600 nanofiltration membranes with a molecular weight cutoff of 600 Da were loaded into two 4″ crossflow ultrafiltration cells in an Evonik METCell Crossflow filtration system. The membrane was conditioned by filling the filtration system with 640 mL of anhydrous ethanol, circulating the ethanol throughout the filtration system while heating the ethanol to a temperature of 50° C. with a hot plate. Ethanol flowed over the retentate side of the membrane at approximately 1 L/min using a gear pump, and the system was pressurized with nitrogen gas to a pressure of 30 barg. The membrane permeated ethanol until 25 mL of ethanol was collected from the cell in order to flush preservatives from the membrane, at which point the system was depressurized and thoroughly drained of conditioning ethanol. 700 mL of the crude ethyl ferulate extract, which contained 1.03 g/L of ethyl ferulate, was loaded into the filtration system as the crude feed. The extract was continuously circulated over the retentate side of the membranes at 1 L/min using a gear pump while heating the fluid inside the system to 50° C. using a hot plate, then the system was pressurized to 30 barg using nitrogen gas. Ethanol containing ferulate permeated through the membrane and was collected in a flask, while some impurities such as proteins and lignin were retained by the membrane and remained within the filtration system. The average permeate flux was 11.4 L/(m² hr). The permeate was collected over several hours and pooled into an aggregate permeate sample. Permeation was stopped and the system was depressurized after 550 mL of permeate was collected. The aggregate permeates for the filtration had a concentration of 4.079 mM compared to the feed concentration of 6.617 mM, corresponding to 62% permeability. The total ferulate recovery for this stage was 48%. A first diafiltration was then carried out to recovery more ferulate by re-diluting the retentate with 550 mL of clean anhydrous ethanol and restoring the solution to its original volume of 700 mL. The extract was then continuously circulated over the retentate side of the membranes at 1 L/min using a gear pump while heating the fluid inside the system to 50° C. using a hot plate, then the system was pressurized to 30 barg using nitrogen gas. The average permeate flux was 11.5 L/(m² hr). The permeate was collected over several hours and pooled into an aggregate permeate sample. Permeation was stopped and the system was depressurized after 590 mL of permeate was collected. The aggregate permeates for the filtration had a concentration of 1.598 mM compared to the feed concentration of 2.132 mM, corresponding to 75% permeability. The ferulate recovery for this stage was 63% of the feed to it, and 81% cumulative with the first filtration. A second diafiltration was then carried out to recovery more ferulate by re-diluting the retentate with 590 mL of clean anhydrous ethanol and restoring the solution to its original volume of 700 mL. The extract was then continuously circulated over the retentate side of the membranes at 1 L/min using a gear pump while heating the fluid inside the system to 50° C. using a hot plate, then the system was pressurized to 30 barg using nitrogen gas. The average permeate flux was 10.6 L/(m² hr). The permeate was collected over several hours and pooled into an aggregate permeate sample. Permeation was stopped and the system was depressurized after 550 mL of permeate was collected. The aggregate permeates for the filtration had a concentration of 0.705 mM compared to the feed concentration of 0.971 mM, corresponding to 73% ferulate permeability. The ferulate recovery for this stage was 57% of the feed to it, and 92% cumulative with the two prior filtrations.

### Example 12

Concentration of ethyl ferulate and ethyl coumarate by nanofiltration. In this example, ultrafiltered ethyl ferulate extract produced using the method in Example 2 was concentrated using nanofiltration. A circular Evonik DuraMem 300, flat-sheet nanofiltration membrane with a molecular weight cutoff of 300 Da was loaded into each of three 4″ crossflow ultrafiltration cells in an Evonik METCell Crossflow filtration system. The membranes were conditioned by filling the filtration system with 640 mL of anhydrous ethanol, circulating the ethanol over the retentate side of the membranes at 1 L/min using a gear pump, and pressurizing the system with nitrogen gas to a pressure of 30 barg. The membranes permeated ethanol until 25 mL of ethanol was collected from each cell, at which point the system was depressurized and the conditioning ethanol was drained. 640 mL of the ultrafiltered ethyl ferulate extract, which contained 3.33 g/L of total dissolved solids and 0.694 g/L of ethyl ferulate, was loaded into the filtration system. The extract was continuously circulated over the retentate side of the membranes at 1 L/min using a gear pump, then the system was pressurized to 50 barg using nitrogen gas. Ethanol and some small molecules such as salts permeated through the membrane, while ferulate, coumarate, and some impurities were retained by the membrane, thereby concentrating the ferulate extract in the retentate by selectively permeating the solvent through the membrane, 10 mL of permeate from each cell was discarded until pseudo-steady-state was reached, then permeate collection was carried out for 1.5 hours, then refilled with 194 mL of ultrafiltered ferulate extract and carried out for another two hours. The average permeate flux was 11.8 L/(m² hr). The permeate samples were pooled into an aggregate permeate sample and 431 mL of retentate was recovered from the filtration system's holding tank. The aggregate permeate had a total dissolved solids content of 0.267 g/L and 0.0275 g/L of ethyl ferulate, corresponding to an ethyl ferulate loss to the permeate of 0.88%. The concentration of ferulate in the retentate increased by a factor of 1.7 times relative to the initial feed.

### Example 13

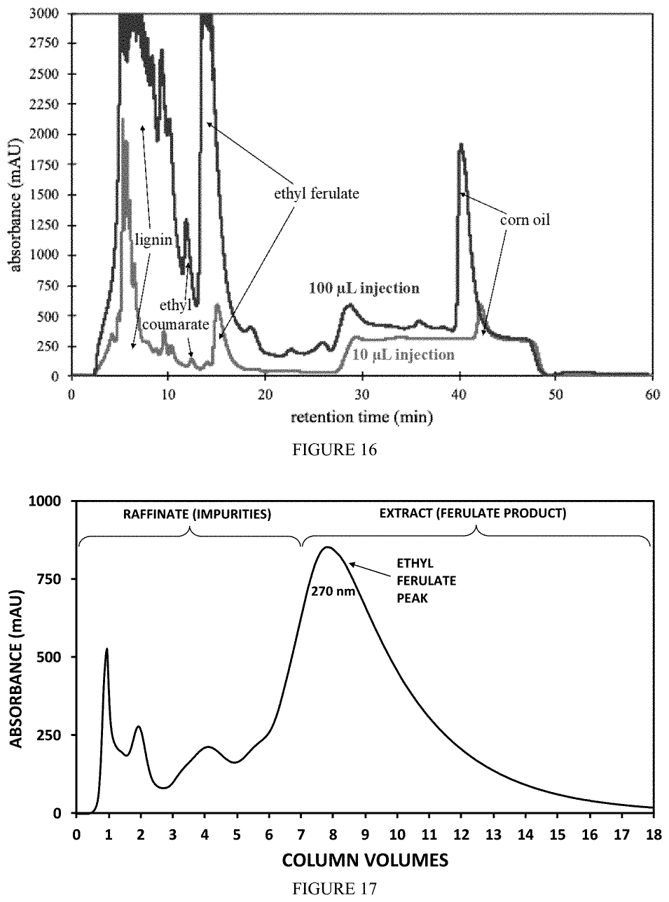

Purification of ethyl ferulate using reverse phase chromatography. In this example, chromatography is used to purify ethyl ferulate from crude extract produced using the method in Example 2. An Agilent 1100 series HPLC was used to carry out the chromatography step. Separation was achieved in an Agilent PL1512-5800 column, which was comprised of a stainless-steel column (dimensions 4.6 mm ID by 250 mm length) packed with poly(styrene/divinylbenzene) resin with average particle size 8 μm and average pore size of 100 Å. The column was held at 25° C. in an isothermal column compartment. The eluent for the chromatographic separation was comprised of 90% ethanol and 10% water by volume. A binary pump flowed eluent at 0.5 mL/min through the column until it reached equilibrium. Then, 10 μL of crude extract containing ethyl ferulate was injected before the column. The run was carried out isocratically for 25 minutes using the 90% ethanol eluent. After 20 minutes and after the ethyl ferulate peak passed the detector—the eluent was ramped to 100% ethanol and the column temperature was ramped to 40° C. over the course of 1 minute. After 19 minutes of 100% ethanol elution and after the corn oil (ethyl oleate and ethyl linoleate) peak passed the detector, the desorption cycle was completed and the pump was ramped to elute with 90% ethanol 10% water while the column temperature was adjusted 25° C. over the course of 1 minute. In a separate experiment, 100 μL of crude extract was injected before the column and eluted using the same method. An ethyl ferulate fraction was collected from the detector outlet as the ethyl ferulate eluted; the fraction was then reinjected into the HPLC using a separate analytical method. The UV-VIS chromatograms for both the 10 μL and 100 μL injections are shown in FIG. 16.

### Example 14

Purification of ethyl ferulate using optimized reverse phase chromatography. In this example, chromatography is used to purify ethyl ferulate from a de-oiled ferulate extract produced via the method of Example 15. The chromatographic purification can occur in a number of ways, but in this example a single GE HiScale 26/50 column was packed with 101 mL of Purolite Chromalite PCG1200M. 1 column volume (CV) was therefore equal to 101 mL in this example. A GE AKTA Explorer 100 FPLC with P-900 pump and 100 Pump heads, UV-900 monitor, pH/C-900, P-960 sample pump, M-925 mixed with 0.6 mL mixing chamber, IV-908 motor valve, PV-908 motor valve, and Frac-950 fraction collector with 250 mL sample bottles was used for the de-oiling process. The composition of the column effluent was analyzed by monitoring UV wavelengths of 205, 270, and 362 nm. Ferulate extract, having been nanofiltered using a method similar to that in Example 11 and de-oiled using a method similar to that in Example 15, was first completely stripped of solvent on a rotary evaporator, yielding a flowable residue. The residue was mixed with 150 mL of ethanol and 150 mL of water to produce a chromatography feed with a solvent composition of approximately 50% ethanol and 50% water by volume. When referring to solvent compositions in this example, the amount of ethanol shall be specified as a volume percent and the balance of the solvent shall be assumed to be water. The column was first flushed with 5 BV of 50% ethanol to prepare for the run. 10 mL of chromatography feed containing ethyl ferulate was injected into the column at a flowrate of 10 mL/min, or 5.9 CV/hr. The eluent for the chromatographic separation was comprised of 50% ethanol and 50% water by volume. A pump flowed eluent at 32 mL/min, or 19 CV/hr through the column. The run was carried out isocratically in this method until 18 CV of eluent were flowed through the column. Automatic UV peak fractionate was used to collect two fractions: a) raffinate containing impurities, and b) extract containing ethyl ferulate and coumarate. The peak fractionation was set to switch to collecting the ferulate fraction when 326 nm absorbance exceeded 1000 mAU, which was determined in other experiments to produce high purity ethyl ferulate with maximum recovery. The UV absorption data and chromatography are shown in FIG. 17. This chromatography step was repeated 37 consecutive times and the ferulate fractions were pooled. The solvent was removed from the ferulate fraction, which was analyzed by HPLC to contain 95% ethyl ferulate and 5% ethyl coumarate.

### Example 15

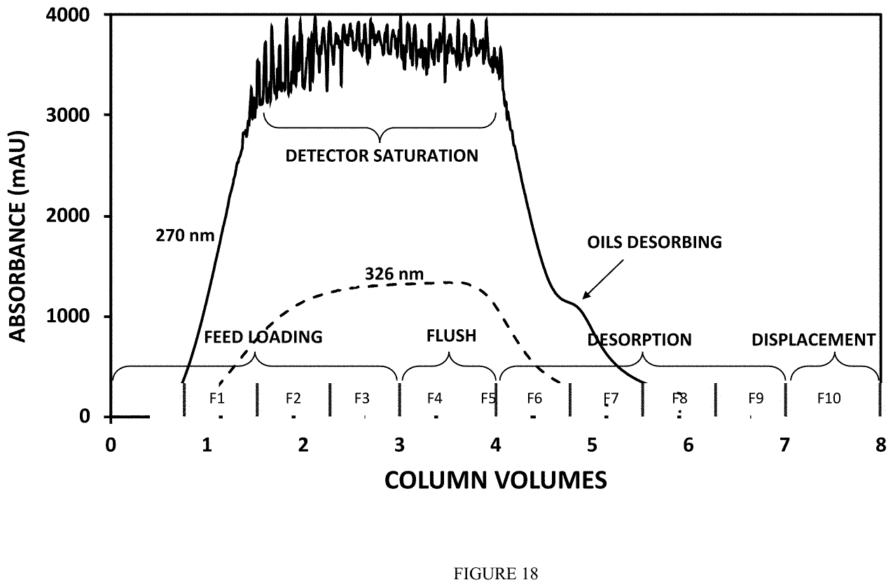

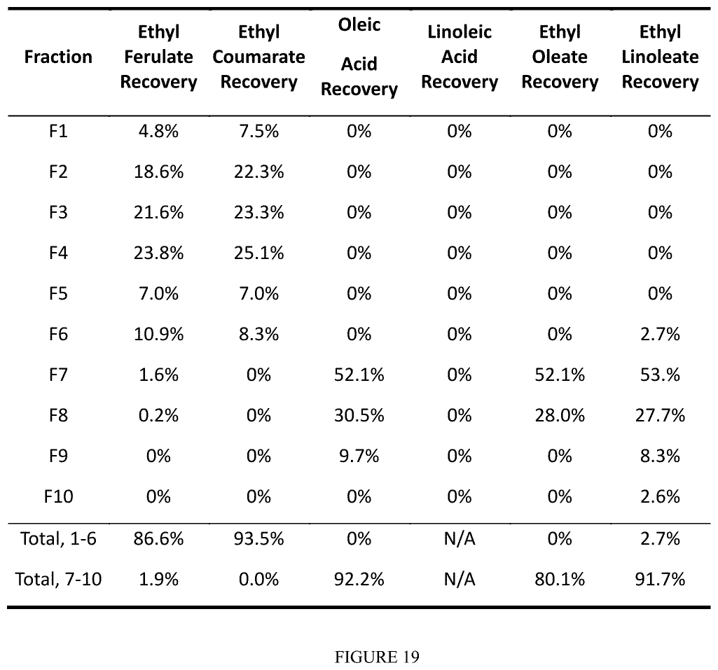

De-oiling ethyl ferulate by absorption of aliphatic oils onto a packed bed of resin. In this example, oils or other aliphatic substances are removed prior to the main chromatography separation. This de-oiling step can occur in a number of ways, but in this example, a single GE XK 26/70 column was packed with 330 mL of Purolite PuroSorb PAD1200, 1 bed volume was therefore equal to 330 mL in this example. A GE AKTA Explorer 100 FPLC with P-900 pump and 100 Pump heads, UV-900 monitor, pH/C-900, P-960 sample pump, M-925 mixed with 0.6 mL mixing chamber, IV-908 motor valve, PV-908 motor valve, and Frac-950 fraction collector with 250 mL sample bottles was used for the de-oiling process. The composition of the column effluent was analyzed by monitoring UV wavelengths of 205, 270, and 362 nm. 985 mL of a ferulate extract, having been nanofiltered via the method of Example 11, was mixed with 110.4 mL water to produce a deoiling feed with a solvent composition of approximately 90% ethanol and 10% water by volume. When referring to solvent compositions in this example, the amount of ethanol shall be specified as a volume percent and the balance of the solvent shall be assumed to be water. The bed was initially washed with 1 L of 90% ethanol. In the loading step, three bed volumes of deoiling feed was percolated through the adsorbent bed at 2 bed volumes per hour. After the feed loading was complete, the column was flushed with flushing solvent consisting of 90% ethanol to remove any leftover ferulate while leaving oils adsorbed to the column. 1 bed volume of flushing solvent was pumped through the column at 2 bed volumes per hour. The effluent of the loading and flushing steps, which contained ferulate and having been de-oiled, was collected into a ferulate fraction in various fraction volumes. Upon completion of flushing, oils or aliphatic compounds adsorbed to the column were removed in a desorption step where 3 bed volumes of anhydrous ethanol were flowed through the column at a rate of 2 bed volumes per hour. The desorbed oils were collected as a separate oil fraction. Upon completion of desorption, 1 bed volume of 90% ethanol was flowed through the column at 2 bed volumes per hour in a displacement step. The desorbent ethanol was displaced with 90% ethanol, and the effluent was collected. UV and fraction collection data are shown in FIG. 18. The fractions were analyzed for ethyl ferulate, ethyl coumarate, oleic acid, linoleic acid, ethyl oleate, and ethyl linoleate. The quantities of each of these compounds is in FIG. 19. Fractions F1-F6 were collected and pooled together as a ferulate fraction which, collectively, contained 86.6% of the ethyl ferulate and 93.5% of the ethyl coumarate originally in the feed material. Fractions F7-F10 contained 92.2% of the oleic acid, 80.1% of the ethyl oleate, and 91.7% of the ethyl linoleate originally in the feed material.

### Example 16

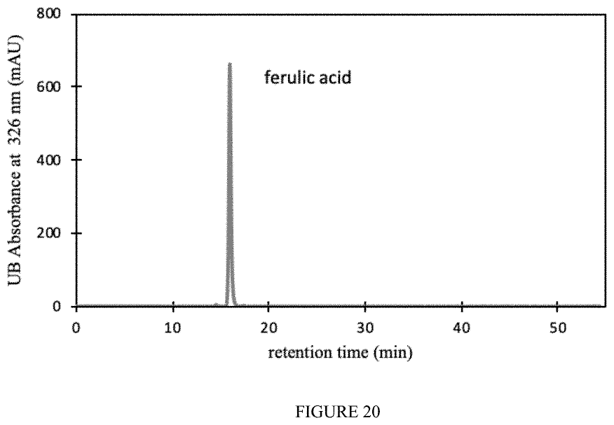

Hydrolysis and crystallization of pure ferulic acid. In this example, ethyl ferulate and ethyl coumarate purified using chromatographic methods was subjected to hydrolysis and crystallization to produce a high-purity ferulic acid product. 1.895 g of semisolid comprised of 78% ethyl ferulate and 2.9% coumaric acid was dissolved in 132 mL of 0.25 M sodium hydroxide at 40° C. by stirring in a 250 mL round-bottom flask and placed in a water bath at 40° C. By maintaining the solution temperature of 40° C. in a water bath for 97 minutes while stirring at 200 rpm with a stir bar, ethyl ferulate and ethyl coumarate were hydrolyzed to the free acids ferulic acid and coumaric acid. The conversion of ethyl ferulate was 100% and the yield of ferulic acid was 96%. Ferulic acid was then precipitated by acidification. 35 mL of 1 M hydrochloric acid was added drop-wise to the stirring solution at 40° C., reaching a final pH of 1. The solution was removed from the water bath, allowed to cool to room temperature, then refrigerated at 4° C. for 24 hours. Solid, crude ferulic acid crystals were recovered by vacuum filtration through a filter paper (retention >7 micron) and the filter cake was dried at 120° C. for three hours. The filter cake was allowed to cool in a desiccator and then weighed. 1.2058 g of crude filter cake was recovered and was comprised of 77.8% ferulic acid and 1.1% coumaric acid. 1.0214 g of the crude filter cake, containing 0.795 g of ferulic acid, was then recrystallized by dissolving in 70 mL of boiling deionized water at 100° C. Once the ferulic acid was completely dissolved, the solution was vacuum filtered twice through filter paper (retention >7 micron) while still at 100° C. in order to remove insoluble impurities. The filtrate was allowed to cool slowly to room temperature and was then refrigerated for 24 hours. Fine ferulic acid crystals were recovered by vacuum filtration through a filter paper (retention >7 micron), rinsed with 25 mL of cold deionized water, then dried at 120° C. for 3.5 hours. The crystals were allowed to cool in a desiccator and weighed. 0.736 g of fine ferulic acid crystals were obtained. HPLC analysis, as shown in FIG. 20, found that the ferulic acid was >98% pure and contained 0.55% coumaric acid by mass.

### Example 17

Hydrolysis of ethyl ferulate purified by reverse phase chromatography and subsequent crystallization of pure ferulic acid. In this example, ethyl ferulate and ethyl coumarate purified using the chromatographic methods of Example 14 were subjected to hydrolysis and crystallization to produce a high-purity ferulic acid product. 267.4 mg of semisolid material comprised of 95.6% ethyl ferulate was dissolved in 22.5 mL of 0.25 M sodium hydroxide at 40° C. by stirring in a 100 mL round-bottom flask and placed in a water bath at 40° C. By maintaining the solution temperature of 40° C. in a water bath for 90 minutes while stirring at 200 rpm with a stir bar, ethyl ferulate and ethyl coumarate were hydrolyzed to the free acids ferulic acid and coumaric acid. After 90 minutes of hydrolysis, the solution was acidified drop-wise with 7 mL of 1 M hydrochloric acid until the pH of the solution was 1-2 as indicated by pH paper. The conversion of ethyl ferulate was 100% and the yield of ferulic acid was 96%. Ferulic acid was then precipitated by acidification. Solid, crude ferulic acid crystals were recovered by vacuum filtration through a filter paper (retention >7 micron) and the filter cake was dried at 105° C. overnight. The filter cake was allowed to cool in a desiccator and then weighed. 167 mg of crude filter cake was recovered and was comprised of 96% ferulic acid and 4% coumaric acid.

### Example 18

Reactive extraction of ferulic acid and ethyl ferulate followed by hydrolysis and ion exchange chromatography. In a prophetic example, a mixture of ethyl ferulate and ferulic acid was extracted from corn fiber using the methods described in Example 6. As described in Example 6, the yield of ethyl ferulate was 0.66% and the yield of ferulic acid was 0.45% with respect to the initial 45 g corn fiber. The ethanol solvent and solids were filtered through a 75 μm mesh filter bag, rinsed with 450 mL of ethanol, and pressed to remove solvent containing ethyl ferulate and ferulic acid from the fiber. After pressing the solids, the solids contained in the filter bag were rinsed with an additional 450 mL ethanol and pressed for a second time to remove solvent containing ethyl ferulate and ferulic acid from the corn fiber. Following removal of the fiber, the liquid reaction product was comprised of a mixture of ferulic acid, ethyl ferulate, corn oil, and impurities including protein and lignin contained in a solvent of mixed ethanol and water. The ethanol was then removed by distillation to leave an aqueous solution containing ferulic acid, ethyl ferulate, corn oil, and impurities including protein and lignin. Solid sodium hydroxide was then added to the aqueous mixture to bring the concentration of sodium hydroxide to in the mixture and a reaction pH 12. The mixture was then heated to 40° C. and held at 40° C. for 4 hours to hydrolyze the ethyl ferulate to ferulic acid before cooling to room temperature. Following hydrolysis the reaction mixture contained ferulic acid, corn oil, and impurities including protein and lignin. The reaction mixture is acidified to pH 7 using 1M hydrochloric acid, then filtered using a filter paper (>7 μm retained) to produce feed for ion exchange. The ferulic acid is then purified using ion exchange. A HiScale 26/40 column is packed with 50 mL of Purolite A5000H, a strong anion exchange resin, and rinsed with deionized water. The feed containing ferulic acid is then percolated through the bed at a flowrate of 2 bed volumes per hour, or 100 mL/hr. As the feed percolates through the ion exchange resin, ferulic acid is adsorbed and bound to the resin as impurities flow freely through the column and exit in the effluent. After all of the feed is loaded, 2 bed volumes of DI are used to wash impurities through the column while leaving the ferulate bound to the resin. Ferulic acid is then displaced from the column by percolating 2 bed volumes of 1 M hydrochloric acid through the column at a flow rate of 2 bed volumes per hour. Hydrochloric acid is washed from the column by flowing 1 bed volume of deionized water through the column at a rate of 2 bed volumes per hour, then the column is regenerated by flowing 2 bed volumes of 1 M sodium hydroxide through the column at a rate of 2 bed volumes per hour. Ferulic acid is then recovered from the displacement solution by evaporative crystallization.

### Example 19

Integrated process to produce high-purity ferulic acid. In this example, corn fiber is pretreated, then ethyl ferulate is reactively extracted from pretreated corn fiber, purified using chromatography, converted to ferulic acid, crystallized, recrystallized, and collected as a high-purity solid. 225 g of corn fiber that was previously dried at 90° C. under vacuum for 50 hr was loaded into a 7.5 L stirred batch reactor (Parr Instruments 4550) with 3.6 L of deionized water. The reactor was sealed and purged with nitrogen (Airgas NI 300) three times by pressurizing the reactor to ca. 6 bar and subsequently venting the pressure to ca. 1 bar. The temperature was increased to 80° C. at a ramp rate of 220° C.^{hr-1} while the reactor was stirred at 200 rpm. The temperature was held at 80° C. for 2 hours before returning to room temperature. After the pretreatment, the water solvent and solids were filtered through a 75 μm mesh filter bag and rinsed with 1 L of deionized water. The pretreated fiber was then dried in an oven at 80° C. for 4 days. The recovered 164.8 g of dried, pretreated fiber was then loaded into a 7.5 L stirred batch reactor (Parr Instruments 4550) with 3.6 L of 0.04 M sodium hydroxide in 200 proof anhydrous ethanol. The reactor was sealed and purged with nitrogen (Airgas NI 300) three times by pressurizing the reactor to ca. 6 bar and subsequently venting the pressure to ca. 1 bar. The temperature was increased to 145° C. at a ramp rate of 220° C.^{hr-1} while the reactor was stirred at 200 rpm. The temperature was held at 145° C. for 4 hours before returning to room temperature. After the reaction, the ethanol solvent and solids were filtered through a 75 μm mesh filter bag and rinsed with 1 L of ethanol. Fine solids were removed from the extract by filtering through filter paper (particle retention >11 μm) and rinsed with 25 mL ethanol. The yield of ethyl ferulate in the filtrate was analyzed by HPLC. The yield of ethyl ferulate was determined to be 1.7% with respect to the 225 g of corn fiber subjected to pretreatment. The purity was determined from the yield of ethyl ferulate and the total dissolved solids in the crude extract. The purity of ethyl ferulate in the crude extract was 12.6% by mass. A chromatography column was prepared by loading 1440 g of 200-300 mesh silica as a slurry in hexane into a cylindrical, glass column with an inner diameter of 100 mm. The total bed depth was 250 mm. 29.2 g of crude extract was then dissolved in ethanol and mixed with 94 g of silica. The ethanol solvent was removed, leaving a free-flowing material comprised of crude extract dry-loaded onto silica. The dry-loaded crude extract on silica was charged to the top of the chromatography column. To start the run, 200 mL of hexanes were added to a solvent reservoir at the top of the column and the column was pressurized with approximately 5 psi of nitrogen. Once the solvent reservoir was emptied, additional solvents were added and eluted in the following order: 200 mL 5%_{v/v} ethyl acetate in hexanes, 200 mL 10%_{v/v} ethyl acetate in hexanes, 200 mL 15%_{v/v} ethyl acetate in hexanes, 3 L 20%_{v/v} ethyl acetate in hexanes, 400 mL 40%_{v/v} ethyl acetate in hexanes. 20 mL fractions were collected throughout the run and analyzed by thin layer chromatography to check for ethyl ferulate and corn oil. Fractions 9-19 contained corn oil, fractions 20-28 contained an ethyl ferulate and corn oil mixture, fractions 29-42 contained ethyl ferulate, and fraction 43-48 contained ethyl ferulate and an unknown impurity. Fractions 29-42 were pooled together and the chromatography solvent was removed, yielding 3.38 g of purified material that was found to be 80% ethyl ferulate by mass using HPLC. 1.895 g of semisolid comprised of 78% ethyl ferulate and 2.9% coumaric acid was dissolved in 132 mL of 0.25 M sodium hydroxide at 40° C. by stirring in a 250 mL round-bottom flask and placed in a water bath at 40° C. By maintaining the solution temperature of 40° C. in a water bath for 97 minutes while stirring at 200 rpm with a stir bar, ethyl ferulate and ethyl coumarate were hydrolyzed to the free acids ferulic acid and coumaric acid. The conversion of ethyl ferulate was 100% and the yield of ferulic acid was 96%. Ferulic acid was then precipitated by acidification. 35 mL of 1 M hydrochloric acid was added drop-wise to the stirring solution at 40° C., reaching a final pH of 1. The solution was removed from the water bath, allowed to cool to room temperature, then refrigerated at 4° C. for 24 hours. Solid, crude ferulic acid crystals were recovered by vacuum filtration through a filter paper (retention >7 micron) and the filter cake was dried at 120° C. for three hours. The filter cake was allowed to cool in a desiccator and then weighed. 1.2058 g of crude filter cake was recovered and was comprised of 77.8% ferulic acid and 1.1% coumaric acid. 1.0214 g of the crude filter cake, containing 0.795 g of ferulic acid, was then recrystallized by dissolving in 70 mL of boiling deionized water at 100° C. Once the ferulic acid was completely dissolved, the solution was vacuum filtered twice through filter paper (retention >7 micron) while still at 100° C. in order to remove insoluble impurities. The filtrate was allowed to cool slowly to room temperature and was then refrigerated for 24 hours. Fine ferulic acid crystals were recovered by vacuum filtration through a filter paper (retention >7 micron), rinsed with 25 mL of cold deionized water, then dried at 120° C. for 3.5 hours. The crystals were allowed to cool in a desiccator and weighed. 0.736 g of fine ferulic acid crystals were obtained. HPLC analysis, as shown in FIG. 20, found that the ferulic acid was >98% pure and contained 0.55% coumaric acid by mass.

Having described various systems and methods herein, certain aspects can include, but are not limited to:

An aspect for the production of ferulic acid including a process for producing ferulic acid from biomass, where said process may comprise any number of the following steps in various orders: pretreatment of biomass by contact with a pretreatment solvent to advantageously extract impurities into the pretreatment solvent liquid, reactive extraction of ferulate from biomass into an extraction solvent and optionally catalyzed by an extraction aid, separation of spent biomass solids from the liquid extract by means of a solid-liquid separation unit, purification of ferulate-containing extract by ultrafiltration or nanofiltration, capture column adsorptive removal of aliphatic oils from the ferulate-containing extract, chromatographic purification of ferulate using simulated moving bed methods, basic aqueous hydrolysis of ferulate to ferulic acid, purification of ferulic acid using ion-exchange chromatography, isolation of solid ferulic acid by precipitation and subsequent filtration from aqueous solution, and recrystallization of ferulic acid from hot aqueous solutions.

In a first aspect, a process for a reactive separation of organic molecules from biomass comprises: a reaction step for the biomass, a simultaneous extraction step using an extraction solvent to solvate products, recovering the products from the reaction and simultaneous extraction steps to produce a crude extract, wherein the products comprise a ferulate or a coumarate; an ultrafiltration step to remove impurities from the crude extract to produce a filtered extract, and a chromatographic separation step to produce purified ferulate or coumarate from the filtered extract.

A second aspect can include the process of the first aspect, wherein the ferulate comprises a ferulic acid ester, and wherein the coumarate comprises a coumaric acid ester.

A third aspect can include the process of the first or second aspect, wherein recovering the products from the reaction and simultaneous extraction steps comprises a coarse filtration step to recover the products.

A fourth aspect can include the process of any one of the first to third aspects, further comprising: a pretreatment step for the biomass using a pretreatment solvent;

A fifth aspect can include the process of any one of the first to fourth aspects, further comprising: a hydrolysis step to convert the ferulate into ferulic acid in solution, a precipitation step to produce solid ferulic acid from the solution, and a recrystallization step to produce high-purity, solid ferulic acid from the solid ferulic acid.

A sixth aspect can include the process of the fourth aspect, wherein the biomass is pretreated to remove impurities using a pretreatment solvent.

A seventh aspect can include the process of the fourth or sixth aspect, wherein the pretreatment solvent comprises 50-100% water and 0-50% any aliphatic alcohols.

An eighth aspect can include the process of the fourth, sixth, or seventh aspects, wherein the pretreatment solvent is 100% water.

A ninth aspect can include the process of the fourth or any one of the sixth to eighth aspects, wherein the pretreatment solvent contains an acid that is hydrochloric acid, sulfuric acid, phosphoric acid, or citric acid.

A tenth aspect can include the process of the fourth or any one of the sixth to ninth aspects, wherein the pretreatment solvent contains a base that is any first or second group hydroxide, carbonate, bicarbonate, or ammonium hydroxide.

An eleventh aspect can include the process of the tenth aspect, wherein the base has a concentration between 0-1 N.

A twelfth aspect can include the process of the any one of the first to eleventh aspects, wherein a mass ratio of extraction solvent:biomass is in a range of 5 to 15

A thirteenth aspect can include the process of the fourth or any one of the sixth to twelfth aspects, wherein the pretreatment step is carried out in a reactor, and wherein the reactor is heated to a temperature of between about 30-100° C.

A fourteenth aspect can include the process of the thirteenth aspect, wherein the reactor is held at the reaction temperature for 0.5-8 hours.

A fifteenth aspect can include the process of the any one of the first to fourteenth aspects, wherein the products are extracted from the biomass in a pressurized stirred batch reactor using a liquid extraction solvent and a base in which the ferulate and the coumarate remain.

A sixteenth aspect can include the process of the fifteenth aspect, wherein a base is any first or second group hydroxide, carbonate, bicarbonate, or ammonium hydroxide.

A seventeenth aspect can include the process of the fifteenth or sixteenth aspect, wherein the reactor contains liquid and a pressurized gas consisting of nitrogen, argon, helium, or hydrogen, or their mixtures.

An eighteenth aspect can include the process of any one of the first to seventeenth aspects, wherein the biomass is obtained from agricultural products.

A nineteenth aspect can include the process of any one of the first to eighteenth aspects, wherein the extraction solvent is 100% water.

A twentieth aspect can include the process of any one of the first to nineteenth aspects, wherein the extraction solvent comprises 50-100% any aliphatic alcohols and 0-50% water.

A twenty first aspect can include the process of any one of the first to twentieth aspects, wherein the extraction solvent is 100% ethanol or 100% methanol.

A twenty second aspect can include the process of any one of the first to twenty first aspects, wherein a mass ratio of extraction solvent:biomass is in a range of 2 to 30.

A twenty third aspect can include the process of any one of the first to twenty first aspects, wherein a mass ratio of extraction solvent:biomass is in a range of 5 to 15.

A twenty fourth aspect can include the process of any one of the fifteenth to seventeenth aspects, wherein the base has a concentration between 0-1 N.

A twenty fifth aspect can include the process of any one of the first to twenty fourth aspects, wherein the reaction step is carried out in a reactor, and wherein the reactor is heated to a reaction temperature of between about 80-250° C.

A twenty sixth aspect can include the process of the twenty fifth aspect, wherein the reactor is held at the reaction temperature for 1-15 hours.

A twenty seventh aspect can include the process of any one of the first to twenty sixth aspects, wherein the solvent comprises water, and wherein the products further comprise ferulic acid, wherein the process further comprises: converting at least a portion of the ferulic acid to an ester using a transesterification reaction, wherein the transesterification reaction occurs prior to the chromatographic separation step.

In a twenty eighth aspect, a process to purify biomass extracts and produce high purity ferulates or coumarates comprises: using liquid chromatography to extract products from a biomass extract solution, wherein the products comprise ferulates and coumarates.

A twenty ninth aspect can include the process of the twenty eighth aspect, wherein the liquid chromatography process comprises a simulated moving bed chromatography system used to separate biomass extractives, the ferulates, and the coumarates into substantially pure fractions.

A thirtieth aspect can include the process of the twenty eighth or twenty ninth aspect, wherein the liquid chromatography process comprises a stationary phase, and wherein the stationary phase is an adsorbent resin comprised of poly(styrene/divinyl benzene), poly(acrylic acid), poly(methacrylic acid), poly(acrylic acid/divinyl benzene), poly(methacrylic acid/divinyl benzene), or an inorganic solid comprised of silica, alumina, or magnesium silicates.

A thirty first aspect can include the process of any one of the twenty eighth to thirtieth aspects, further comprising: removing, using ultrafiltration, impurities from the biomass extract solution prior to passing the biomass extract solution to the liquid chromatography.

A thirty second aspect can include the process of the thirty first aspect, wherein the ultrafiltration uses an ultrafiltration membrane, and wherein the ultrafiltration membrane has a molecular weight cutoff of 300-10,000 Daltons.

A thirty third aspect can include the process of the thirty first or thirty second aspect, wherein the ultrafiltration is carried out with a diafiltration step.

A thirty fourth aspect can include the process of any one of the twenty eighth to thirty third aspects, wherein using the liquid chromatography is preceded by nanofiltration as a means of concentrating the feed to the chromatography system.

A thirty fifth aspect can include the process of the thirty fourth aspect, wherein the nanofiltration uses a nanofiltration membrane, and wherein the nanofiltration membrane has a molecular weight cutoff of 50-300 Daltons.

A thirty sixth aspect can include the process of any one of the twenty eighth to thirty fifth aspects, wherein the liquid chromatography process uses a mobile phase or eluent, and wherein the mobile phase or eluent used in the liquid chromatography comprises 0-30% water with a balance of one or more aliphatic alcohols.

A thirty seventh aspect can include the process of any one of the twenty eighth to thirty sixth aspects, wherein the chromatography is carried out at a temperature between 20-100° C.

A thirty eighth aspect can include the process of any one of the twenty eighth to thirty seventh aspects, further comprising: combining the products with an aqueous base solution to form a hydrolysis mixture; heating the hydrolysis mixture; and hydrolyzing any ferulates to ferulic acid in the hydrolysis mixture in response to the heating.

A thirty ninth aspect can include the process of the twenty eighth aspect, wherein the ferulate is ethyl ferulate.

A fortieth aspect can include the process of the thirty eighth or thirty ninth aspect, wherein the base is any first or second group hydroxide, carbonate, bicarbonate, ammonium hydroxide, or any combination thereof and has a concentration between 0-1 N.

A forty first aspect can include the process of any one of the thirty eighth to fortieth aspects, wherein the hydrolysis mixture is heated to 40-80° C.

A forty second aspect can include the process of any one of the thirty eighth to forty first aspects, wherein after an appropriate dwell time the hydrolysis mixture is acidified with an aqueous acid to a pH of 0.5-4 and ferulic acid is precipitated.

A forty third aspect can include the process of the forty second aspect, wherein the aqueous acid is hydrochloric acid, nitric acid, phosphoric acid, sulfuric acid, acetic acid, citric acid, or any combination thereof and has a concentration between 0-10 N.

A forty fourth aspect can include the process of any one of the thirty eighth to forty third aspects, further comprising: producing crude solid or semisolid ferulic acid based on the hydrolyzing; dissolving the crude solid ferulic acid in an aqueous solution to form dissolved ferulic acid; heating the dissolved crude ferulic acid; filtering the dissolved crude ferulic acid; removing one or more insoluble impurities from the dissolved ferulic acid using the filtering; decanting any oil phase from the aqueous solution; and cooling the dissolved ferulic acid after removing the one or more insoluble impurities to produce purified solid ferulic acid.

A forty fifth aspect can include the process of any one of the first to twenty seventh aspects, wherein the products are extracted from the biomass in a pressurized continuous stirred-tank reactor using a liquid extraction solvent containing a base in which the ferulate and the coumarate remain.

A forty sixth aspect can include the process of the forty fifth aspect, wherein the biomass and liquid extraction solvent are fed to the continuous stirred-tank reactor as a slurry.

A forty seventh aspect can include the process of the forty fifth aspect, wherein the biomass is obtained from agricultural products.

A forty eighth aspect can include the process of any one of the forty fifth to forty seventh aspects, wherein the extraction solvent comprises 50-100% any aliphatic alcohols and 0-50% water.

A forty ninth aspect can include the process of any one of the forty fifth to forty eighth aspects, wherein the extraction solvent is 100% ethanol or 100% methanol.

A fiftieth aspect can include the process of any one of the forty fifth to forty ninth aspects, wherein a mass ratio of extraction solvent:biomass is in a range of 2 to 30.

A fifty first aspect can include the process of any one of the forty fifth to fiftieth aspects, wherein a mass ratio of extraction solvent:biomass is in a range of 5 to 15.

A fifty second aspect can include the process of any one of the forty fifth to fifty first aspects, wherein the base is any first or second group hydroxide, carbonate, bicarbonate, or ammonium hydroxide and has a concentration between 0-1 N.

A fifty third aspect can include the process of any one of the forty fifth to fifty second aspects, wherein the continuous stirred-tank reactor operates at a steady-state reaction temperature of between about 80-250° C.

A fifty fourth aspect can include the process of any one of the forty fifth to fifty third aspects, wherein the continuous stirred-tank reactor operates with a residence time of 1-15 hours.

A fifty fifth aspect can include the process of any one of the first to twenty seventh aspects, wherein the reaction and simultaneous extraction steps occur in a packed bed reactor, wherein the agricultural biomass acts as a stationary bed.

A fifty sixth aspect can include the process of the fifty fifth aspect, wherein the biomass is obtained from agricultural products.

A fifty seventh aspect can include the process of the fifty fifth or fifty sixth aspect, wherein the extraction solvent comprises 50-100% any aliphatic alcohols and 0-50% water.

A fifty eighth aspect can include the process of any one of the fifty fifth to fifty seventh aspects, wherein the extraction solvent is 100% ethanol or 100% methanol.

A fifty ninth aspect can include the process of any one of the fifty fifth to fifty eighth aspects, wherein the base is any first or second group hydroxide, carbonate, bicarbonate, or ammonium hydroxide and has a concentration between 0-1 N.

A sixtieth aspect can include the process of any one of the fifty fifth to fifty ninth aspects, wherein the packed bed reactor is pressurized to between about 1-30 bar.

A sixty first aspect can include the process of any one of the fifty fifth to sixtieth aspects, wherein a temperature of the packed bed reactor is heated to a reaction temperature of between about 100-250° C.

A sixty second aspect can include the process of the sixty first aspect, wherein the reactor is held at a desired reactor temperature for 0.25-24 hours.

A sixty third aspect can include the process of any one of the fifty fifth to sixty second aspects, wherein after an appropriate dwell time the reactor is cooled to 20° C. or below, and a solvent flow rate is increased to flush the packed bed.

A sixty fourth aspect can include the process of any one of the first to sixty third aspects, wherein after the reactive extraction of ferulate and coumarate the biomass is re-extracted with extraction solvent to extract lignin.

A sixty fifth aspect can include the process of the sixty fourth aspect, wherein the lignin is extracted from the biomass in a pressurized reactor using a liquid extraction solvent in which lignin remains.

A sixty sixth aspect can include the process of the sixty fourth aspect, wherein the biomass is obtained from agricultural products.

A sixty seventh aspect can include the process of the sixty fourth aspect, wherein the extraction solvent comprises 50-100% any aliphatic alcohols and 0-50% water.

A sixty eighth aspect can include the process of any one of the sixty fourth to sixty seventh aspects, wherein a mass ratio of extraction solvent:biomass is in a range of 2:1 to 30:1.

A sixty ninth aspect can include the process of any one of the sixty fourth to sixty seventh aspects, wherein a mass ratio of extraction solvent:biomass is in a range of 5:1 to 15:1.

A seventieth aspect can include the process of any one of the sixty fourth to sixty ninth aspects, wherein the reaction step is carried out in a reactor, and wherein the reactor is heated to a reaction temperature of between about 100-250° C.

A seventy first aspect can include the process of the seventieth aspect, wherein the reactor is operated with a residence time of 1-15 hours.

Embodiments are discussed herein with reference to the Figures. However, those skilled in the art will readily appreciate that the detailed description given herein with respect to these figures is for explanatory purposes as the systems and methods extend beyond these limited embodiments. For example, it should be appreciated that those skilled in the art will, in light of the teachings of the present description, recognize a multiplicity of alternate and suitable approaches, depending upon the needs of the particular application, to implement the functionality of any given detail described herein, beyond the particular implementation choices in the following embodiments described and shown. That is, there are numerous modifications and variations that are too numerous to be listed but that all fit within the scope of the present description. Also, singular words should be read as plural and vice versa and masculine as feminine and vice versa, where appropriate, and alternative embodiments do not necessarily imply that the two are mutually exclusive.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims may be formulated in this Application or of any further Application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any Claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

What is claimed is:

1. A process for a reactive extraction and subsequent purification of organic molecules from biomass comprising:
extracting one or more products from the biomass using an extraction solvent to solvate the products;
contacting the biomass with a reactant during the extracting;
recovering the one or more products, wherein the one or more products comprise extracted organic molecules comprising a ferulate or a coumarate, wherein the one or more products are separated from the biomass as a liquid extract,
performing ultrafiltration or nanofiltration to remove impurities from the one or more products to produce a filtered extract,
extracting oils in the filtered extract to produce a de-oiled extract,
performing transesterification or hydrolysis on the de-oiled extract, wherein the ferulate or the coumarate are reacted in the transesterification or hydrolysis to produce ferulic acid, coumaric acid, ferulate, coumarate, or a combination thereof;
performing adsorptive purification on the ferulic acid, coumaric acid, ferulate, coumarate, or a combination thereof, wherein ferulic acid, coumaric acid, ferulate, coumarate, or a combination thereof are purified to produce one or more purified products.

2. (canceled)

3. The process of claim 1, wherein recovering the one or more products comprises a coarse filtration, centrifugation, or solid-liquid separation step to recover the products.

4. (canceled)

5. The process of claim 1, further comprising:
performing hydrolysis on the de-oiled extract to convert the ferulate into ferulic acid in a solution;
performing a precipitation to produce solid ferulic acid from the solution; and
performing recrystallization on the solid ferulic acid to produce a high-purity, solid ferulic acid from the solid ferulic acid.

6.-10. (canceled)

11. The process of claim 1, wherein the one or more products are extracted from the biomass in a reactor, wherein the one or more products are recovered from the reactor in the extraction solvent and the reactant, and wherein the reactant comprises a base that is any first or second group hydroxide, carbonate, bicarbonate, or ammonium hydroxide and the base has a concentration between 0-1 N.

12. The process of claim 1, wherein the biomass comprises miscanthus, corn bran, corn fiber, corn gluten feed, distillers' grain, corn stover, corn gluten meal, beet fiber, rice hulls, or combinations thereof.

13. The process of claim 1, wherein the extraction solvent is 100% water.

14. The process of claim 1, wherein the extraction solvent comprises 50-100% of one or more aliphatic alcohols and 0-50% water and wherein at least a portion of the water is supplied by the biomass.

15.-16. (canceled)

17. The process of claim 1, wherein a mass ratio of the extraction solvent:biomass is in a range of 2 to 30.

18. The process of claim 1, wherein the extracting and contacting are carried out in a reactor, and wherein the reactor is heated to a reaction temperature of between about 80-250° C. and held at the reaction temperature for 0.25-24 hours.

19. The process of claim 1, wherein the one or more products are extracted from the biomass in a batch reactor using the extraction solvent containing a base, and wherein the one or more products are recovered in the extraction solvent from the pressurized batch reactor.

20. The process of claim 1, wherein extracting and contacting are performed in a continuous stirred-tank reactor, wherein the one or more products are recovered in the extraction solvent comprising the reactant, and where the biomass and extraction solvent are fed to and removed from the continuous stirred-tank reactor as a slurry.

21.-25. (canceled)

26. The process of claim 1, wherein the extraction solvent comprises water and ethanol, and wherein the one or more products further comprise ferulic acid, or a mixture of ferulic acid and ferulate esters, wherein the process further comprises:
removal of ethanol from the extraction solvent,
converting at least a portion of ferulate ester to ferulic acid using a hydrolysis reaction.

27. The process of claim 1 to purify biomass extracts containing ferulate or coumarate comprising:
obtaining the liquid extract from the biomass as a biomass extract; and
performing filtration on the biomass extract to remove large molecular weight impurities from the biomass extract, wherein the filtration uses ultrafiltration or nanofiltration and wherein the ultrafiltration membrane has a molecular weight cutoff of 300-10,000 Daltons.

28.-29. (canceled)

30. The process of claim 27, wherein the filtration is carried out in multiple stages with one or more diafiltration steps, wherein 0.2-2 diafiltration volumes of clean solvent are used in each diafiltration step.

31. A process to selectively remove oil from biomass extracts comprising:
obtaining a biomass extract, wherein the biomass extract is obtained from biomass, and wherein the biomass extract contains a ferulate or a coumarate and one or more oils;
performing liquid-solid adsorption to remove at least a portion of the one or more oils onto a solid phase adsorbent to produce a purified product.

32. The process of claim 31, wherein the solid phase adsorbent is a polymeric resin adsorbent comprised of polystyrene, polystyrene crosslinked with divinyl benzene, octadecylsilane-bonded silica, octylsilane-bonded silica, polyvinylpyrrolidone, polymethacrylate, or other polymeric adsorbent.

33.-36. (canceled)

37. A chromatographic adsorption process to separate products of ferulic acid or ferulate from a biomass extract solution comprising:
providing a biomass extract, wherein the biomass extract comprises the products comprising ferulates, coumarates, and oils;
performing chromatographic adsorption on the products, wherein the chromatographic adsorption occurs in a simulated moving bed chromatography system used to separate products and other biomass extractives into substantially pure fractions, wherein the simulated moving bed chromatography system comprises a stationary phase, and wherein the stationary phase is an adsorbent resin comprised of poly(styrene/divinyl benzene), poly(acrylic acid), poly(methacrylic acid), poly(acrylic acid/divinyl benzene), poly(methacrylic acid/divinyl benzene), or an inorganic solid comprised of silica, alumina, or magnesium silicates.

38.-41. (canceled)

42. The process of claim 37, wherein the ferulates and the coumarates are converted to ferulic acid by a process comprising:
combining the ferulates and the coumarates with an aqueous solution of base to form a hydrolysis mixture;
heating the hydrolysis mixture; and
hydrolyzing at least a portion of the ferulates and the coumarates to ferulic acid in the hydrolysis mixture in response to the heating.

43.-46. (canceled)

47. The process of claim 42, further comprising:
producing crude solid or semisolid ferulic acid based on the hydrolyzing;
dissolving the crude solid ferulic acid in an aqueous solution to form dissolved ferulic acid;
heating the dissolved crude ferulic acid;
filtering the dissolved crude ferulic acid;
removing one or more insoluble impurities from the dissolved ferulic acid using the filtering;
decanting any oil phase from the aqueous solution; and
cooling the dissolved ferulic acid after removing the one or more insoluble impurities to produce purified solid ferulic acid.

48.-51. (canceled)

52. The process of claim 1, wherein the ferulic acid, coumaric acid, ferulate, coumarate, or a combination thereof comprises ferulic acid, and wherein the ferulic acid is purified with an ion exchange process comprising:
adsorption of the ferulic acid to a strong or weak anion exchange resin by the formation of the ionic bonds between the carboxylic acid molecule and exchange sites on the resin,
washing the resin with a liquid such as water or solvents, including ethanol, to remove unbound contaminants that are trapped in the ion exchange resins.
displacement of the ferulic acid from the resin by exchanging the adsorbed carboxylic ion for a stronger, inorganic ion, thereby causing the release of the ferulic acid,
preparation of the anion exchange resin for further cycles of ferulic acid adsorption by regenerating the anion exchange sites on the resin by treating the resin with a strong base, which causes the inorganic anion to be exchanged for a hydroxide ion.
